(19) United States
(12) Patent Application Publication
(10) Pub. No.: US 2017/0348331 A1
(43) Pub. Date: Dec. 7, 2017

(54) METHODS AND COMPOSITIONS FOR MODULATING SEROTONIN LEVELS

(71) Applicant: Children's Hospital & Research Center at Oakland, Oakland, CA (US)

(72) Inventors: Bruce AMES, Berkeley, CA (US); Rhonda PATRICK, Oakland, CA (US)

(73) Assignees: Children's Hospital & Research Center at Oakland, Oakland, CA (US); Children's Hospital & Research Center at Oakland, Oakland, CA (US)

(21) Appl. No.: 15/035,730

(22) PCT Filed: Nov. 11, 2014
(86) PCT No.: PCT/US14/06542

§ 371 Date: May 10, 2016

Related U.S. Application Data
(60) Provisional application No. 61/903,113, filed on Nov. 12, 2013.
(60) Provisional application No. 61/936,110, filed on Feb. 5, 2014.

(51) Int. Cl. A61K 31/59; A61K 9/20; A61K 31/202; A61K 31/405; A61K 9/00; A61K 31/675; A61K 31/519; C07K 16/40; G01N 33/573

(52) U.S. Cl. CPC A61K 31/59; C07K 16/40; A61K 9/20; A61K 31/202; G01N 33/573; C12Y 114/16004; A61K 9/0053; A61K 31/675; A61K 31/519; A61K 31/405; G01N 2333/90245; G01N 2800/28; G01N 2800/50

(57) ABSTRACT

The present application in one aspect provides methods and compositions that are fine-tuned to modulate brain and peripheral serotonin levels, which in turn would lead to prevention and treatment of various diseases associated with a reduced level of brain serotonin. In another aspect, there are provided methods of assessing the peripheral or placenta level of serotonin and associated risks based on the peripheral placental level of TPH1.

### RELATED APPLICATIONS

This application claims priority benefit to U.S. Provisional Application No. 61/903,113, filed on Nov. 12, 2013, and U.S. Provisional Application No. 61/936,110, filed on Feb. 5, 2014, the content of each of which is incorporated herein in their entirety.

### BACKGROUND

Serotonin (5-hydroxytryptamine), a neurotransmitter and brain morphogen, promotes pro-social behavior and correct assessment of emotional social cues. Crockett, 2009, Annals of the New York Academy of Sciences 1167:76-86. Serotonin is synthesized in two steps from tryptophan, an essential amino acid present in small amounts in dietary protein. Step 1: tryptophan hydroxylase (TPH), the rate-limiting enzyme in serotonin synthesis, uses tetrahydrobiopterin (BH4) and iron as cofactors to hydroxylate tryptophan to 5-hydroxytryptophan. Step 2: 5-hydroxytryptophan is decarboxylated to serotonin by aromatic amino acid decarboxylase, a pyridoxal phosphate-requiring enzyme. Walther et al., 2003, Biochem. Pharmacol 66(9):1673-1680. There are two separate tryptophan hydroxylase proteins that are produced by different genes, TPH1 and TPH2, which are localized in different tissues. TPH1 is found in non-brain tissues including the gut enterochromaffin cells, pineal gland, placenta, and T-cells, and it is responsible for producing most of the serotonins found in the body, including the blood. Gutknecht et al. 2009, European Neuropsychopharmacology: the Journal of the European College of Neuropsychopharmacology 19(4):266-282; Leon-Ponte et al., 2007, Blood 109(8):3139-3146; Bonnin et al. 2011, Nature 472(7343):347-350. Almost all of the serotonin in the blood is located in platelets, which do not synthesize serotonin, but instead take it up from the gut pool. Chen 2001, The Journal of Neuroscience: the official journal of the Society of Neuroscience 21(16):6348-6361. TPH2 is entirely restricted to neurons of the raphe nuclei and the enteric nervous system and is the enzyme responsible for producing all of the serotonin in the brain. Gutknecht et al. 2009.

Supplementation with 5-hydroxytryptophan (5-HTP), which crosses the blood-brain barrier, has been used to increase brain serotonin levels. However, the conversion of 5-HTP into serotonin may immediately occur in the GI tract, thus, lowering the bioavailability of 5-HTP to be transported into the brain. Additionally, the conversion of 5-HTP into serotonin in the GI tract is known to cause inflammation in the GI tract and there have been negative GI problems associated with 5-HTP supplementation.

Vitamin D is a fat-soluble vitamin that is converted to calcitriol, a steroid hormone that regulates the expression of approximately 900 different genes, a large number of which impact brain development and function. Chugani et al. 1999, Annals of Neurology 45(3):287-295; Bennett-Clarke et al. 1994, The Journal of Neuroscience: the official journal of the Society for Neuroscience 14(12):7594-7607. The primary source of vitamin D is from skin exposure to UVB radiation emitted from the sun, which induces the subcutaneous synthesis of vitamin D from endogenous 7-dehydrocholesterol. Both sunscreen lotion and melanin, the brown pigment found in skin, block UVB radiation and thus impair the ability of the skin to synthesize vitamin D. A modest amount of vitamin D can be obtained through dietary sources, such as seafood, which is its relatively richest dietary source. The CDC (Center of Drug Control) has reported that vitamin D (30-80 ng/ml) has decreased between 1994-2004 from approximately 60% to 30% in Caucasians, from 10% to 5% in African Americans, and from 24% to 6% in Latinos, indicating that more than half of the US population has insufficient levels of this critical vitamin D hormone. Kennel et al., Mayo Clinic Proceedings, Mayo Clinic 2010, 85(8):752-757; Ginde et al., 2009, Archives of Internal Medicine 169(6):626-632. Serum vitamin D levels have been shown to be lower in autistic compared to non-autistic individuals, and lower serum vitamin D was associated with autistic spectrum disorder severity. Cannel 2008, Medical Hypothesis 70(4):750-759; Grant 2009, Dermatoendocrinol. 1(4):223-228. However, no mechanism or causal link between vitamin D and autistic spectrum disorder has been provided.

### SUMMARY OF THE INVENTION

The present invention in one aspect provides a method of increasing brain serotonin level or treating a brain dysfunction disorder in an individual, comprising administering (such as orally administering) to the individual an effective amount of vitamin D and an effective amount of tryptophan. In some embodiments, there is provided a method of increasing brain serotonin level in an individual, comprising administering (such as orally administering) to the individual an effective amount of vitamin D and an effective amount of tryptophan, wherein the amount of the vitamin D is at least about 500 IU per day, and the amount of the tryptophan is at least about 100 mg per day. In some embodiments, there is provided a method of treating a brain dysfunction disorder in an individual, comprising administering (such as orally administering) to the individual an effective amount of vitamin D and an effective amount of tryptophan, wherein the amount of the vitamin D is at least about 500 IU per day, and the amount of the tryptophan is at least about 100 mg per day. In some embodiments, there is provided a method of improving pro-social behavior or cognitive function in an individual, comprising administering (such as orally administering) to the individual an effective amount of vitamin D and an effective amount of tryptophan, wherein the amount of the vitamin D is at least about 500 IU per day, and the amount of the tryptophan is at least about 100 mg per day. In some embodiments according to any of the methods above, the amount of the vitamin D is between about 500 IU per day to about 6000 IU per day. In some embodiments according any of the methods above, the vitamin D and the tryptophan are administered simultaneously (for example in the same composition). In some embodiments according to any of the methods above, the method further comprises administering to the individual one or more of: vitamin B6, BH4 (tetrahydrobiopterin), omega-3 long chain polyunsaturated fatty acid (LCPUFA), and iron. In some embodiments, the method comprises administering to the individual LCPUFA, for example at the dose of at least about 500 mg. In some embodiments according to any of the methods above, the method further leads to reduced inflammation in the GI tract. In some embodiments according to any of the methods above, the method further leads to an increase of oxytocin level. In some embodiments according to any of the methods above, the individual is human. In some embodiments, the individual is younger than 12 years old. In some embodiments, the individual is a pregnant individual. In some embodiments, the individual has a high peripheral TPH1 level. In some embodiments, the individual has a high placenta TPH1 level.

In another aspect, there is provided a composition comprising vitamin and amino acid, wherein at least about 5% of the vitamin in the composition is vitamin D, wherein at least about 30% of the amino acid in the composition is tryptophan. In some embodiments, the composition further comprises one or more of: vitamin B6, BH4, omega-3 long chain polyunsaturated fatty acid (LCPUFA), and iron. In some embodiments, the unit dose of vitamin D in the composition is at least about 500 IU (such as about 500 IU to about 6000 IU). In some embodiments, the unit dose of tryptophan in the composition is at least about 100 mg (such as 0.5-6 grams).

In another aspect, there is provided a method of assessing the brain serotonin level in an individual, comprising determining the peripheral TPH1 level in the individual using an antibody recognizing TPH1, wherein a high TPH1 level is indicative that the individual has a low brain serotonin level. In some embodiments, there is provided a method of assessing the risk of developing a brain dysfunction disorder in an individual using an antibody recognizing TPH1 using an antibody recognizing TPH1, comprising determining the peripheral TPH1 level in the individual using an antibody recognizing TPH1, wherein a high TPH1 level is indicative that the individual has a low brain serotonin level. In some embodiments according to any of the methods above, the method further comprises administering to the individual an agent that increases brain serotonin level. In some embodiments according to any of the methods above, the method further comprise comprising administering to the individual an effective amount of vitamin D.

In another aspect, there is provided a method of assessing the placenta serotonin level in a pregnant individual, comprising determining the placenta TPH1 level in the individual, wherein a high TPH1 level is indicative that the individual has a high placenta serotonin level. In some embodiments, there is provided a method of assessing the risk of a pregnant individual having a child who develops a brain dysfunction disorder, comprising determining the placenta TPH1 level in the individual, wherein a high TPH1 level is indicative of the risk. In some embodiments, the method further comprises administering to the individual an effective amount of vitamin D.

In another aspect, there is provided a kit comprising a) an agent that increases brain serotonin level; and b) an agent for determining the level of TPH1 (such as an antibody recognizing TPH1).

Also provided are unit dosage forms and articles of manufacture useful for any one of the methods described herein.

All references provided herein are hereby incorporated by reference in their entirety.

### BRIEF DESCRIPTION OF THE FIGURES

FIG. 1 provides representative DR3 vitamin D response element (VDRE) subtypes in TPH1, TPH2, Oxytocin, Oxytocin receptor, and vasopressin receptors AVPR1A and AVPR1B.

FIG. 2 provides a model of the maternal contribution to autoimmune antibodies in the fetal brain. Ab denotes maternal autoantibodies.

### DETAILED DESCRIPTION

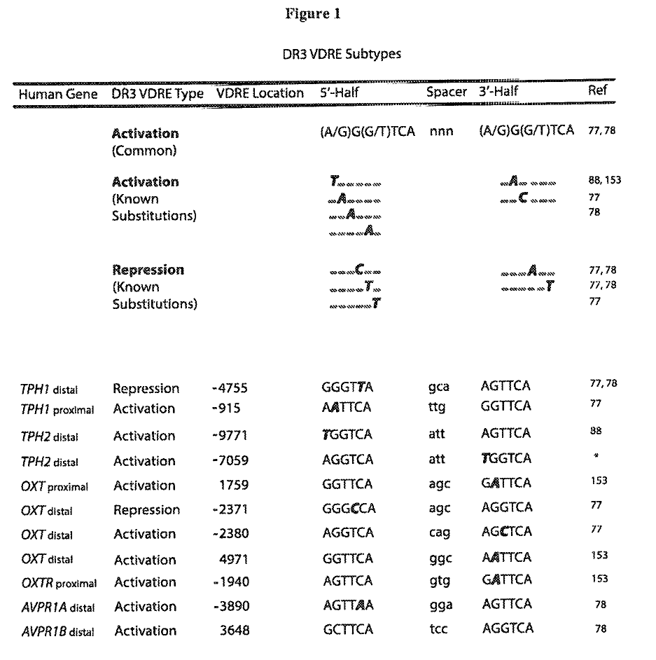

The present invention is based on the surprising discovery that adequate levels of vitamin D are necessary for serotonergic signals during neurodevelopment. By examining the specific sequences of vitamin D regulatory elements (VDRE) found in TPH1 and TPH2, we uncovered that TPH2 has optimum sequences for transcriptional activation, whereas the VDRE in TPH1 has the exact base substitution in the 5′ half site that is only associated with gene repression (FIG. 1). This finding reveals a novel mechanism by which vitamin D transcriptionally represses TPH1 and activates TPH2, thereby inversely affecting serotonin production in peripheral tissue (non-brain tissue) relative to production in the brain.

Furthermore, it was found that vitamin D insufficiency could lead to overexpression of TPH1 in the placenta. This could in turn cause an imbalance in tryptophan catabolism in the placenta, resulting in too much serotonin and too little kynurenine, leading to an autoimmune response attacking the fetus and fetal brain, increasing the risk of the newborn having autistic spectrum disorder and other brain dysfunction disorders.

The present application thus in one aspect provides nutraceutical and pharmaceutical compositions that are fine-tuned to modulate brain and peripheral serotonin levels, which in turn would lead to prevention and/or treatment of various disorders associated with a reduced level of serotonin in the brain and elevated levels in the gut. In another aspect, there are provided methods of increasing brain serotonin level and treating/preventing various disorders associated with a reduced level of brain serotonin by administering effective amounts of vitamin D and tryptophan. In another aspect, there are provided methods of determining brain serotonin levels in an individual and associated risks based on the peripheral level of TPH1. In another aspect, there are provided methods of assessing the placenta (or peripheral) level of serotonin in a pregnant individual and associated risks based on the placental (or peripheral) level of TPH1.

### Definitions

As used herein, “treating” or “treatment” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, and/or increasing quality of life.

The term “individual” refers to a mammal and includes, but is not limited to, human, bovine, horse, feline, canine, rodent, or primate. In some embodiments, the individual is human.

The term “simultaneous administration,” as used herein, means that the first compound and the second compound are administered with a time separation of no more than about 15 minutes, such as no more than about any of 10, 5, or 1 minutes. When the first and second compounds are administered simultaneously, the first and second compound can be contained in the same composition (e.g., a composition comprising both vitamin D and tryptophan), or in separate compositions.

As used herein, the term “sequential administration” means that the first and second compounds are administered with a time separation of more than about 15 minutes, such as more than about 20, 30, 40, 50, 60, or more minutes. Either the first compound or the second compound may be administered first. The first and second compounds are contained in separate compositions, which may be contained in the same or different packages or units.

An individual who “may be suitable,” which includes an individual who is “suitable” for treatment(s) described herein, is an individual who is more likely than not to benefit from administration of said treatment(s). Conversely, an individual who “may not be suitable,” which includes an individual who is “unsuitable” for treatment(s) described herein, is an individual who is more likely than not to fail to benefit from administration of said treatment(s).

It is understood that aspects and embodiments of the invention described herein include “consisting of” and “consisting essentially of” aspects and embodiments.

Reference to “about” a value or parameter herein includes (and describes) variations that are directed to that value or parameter per se. For example, description referring to “about X” includes description of “X.”

As used herein and in the appended claims, the singular forms “a,” “or,” and “the” include plural referents unless the context clearly dictates otherwise.

The composition and methods of the present invention may be substantially free of a specific ingredient described herein. In this context, the term “substantially free” means that the compositions comprise less than about 2%, including less than about 0.5%, less than about 0.1%, or 0%, by weight of the specific ingredient.

### Methods of Administering Vitamin D and Tryptophan

The present application in one aspect provides methods of modulating serotonin levels or treating/preventing certain disorders in an individual, comprising administering (such as orally administering) to the individual an effective amount of vitamin D and an effective amount of tryptophan. In some embodiments, the vitamin D and the tryptophan are administered separately. In some embodiments, the vitamin D and the tryptophan are administered simultaneously. In some embodiments, the vitamin D and the tryptophan are administered in a single composition, such as the vitamin D/tryptophan composition described herein.

The present application provides a mechanism for how vitamin D differentially regulates serotonin levels in the brain versus tissues outside the blood brain barrier or peripheral to the blood brain barrier, herein referred to as the peripheral tissues. We have identified vitamin D response elements (VDRE) on two different tryptophan hydroxylase genes that are functionally opposite to one another; one which induces transcriptional activation of tryptophan hydroxylase 2 (TPH2) by vitamin D in the brain, and one which induces repression of tryptophan hydroxylase 1 (TPH1) in peripheral tissues.

The level of serotonin in the brain depends on the blood levels of its precursor tryptophan, which, unlike serotonin, crosses the blood brain barrier. Tryptophan is a rare amino acid that competes for transport into the brain with the branched chain amino acids, which are more abundant and preferentially transported into the brain. Elevated TPH1 expression, for example due to a lowered vitamin D level, may result in an excess of serotonin in the peripheral tissues, consuming most of the dietary tryptophan, which could further lower its availability to be transported into the brain. Our findings reported herein suggests a potential synergistic role between vitamin D and tryptophan on boosting the serotonin level in the brain and decreasing the serotonin level in peripheral tissues, such as the gastrointestinal tract (GI tract) and the blood. Supplementation of tryptophan would synergize with vitamin D to boost brain serotonin and promote pro-social behavior, focus and attention, sensory gathering, improved mood, and lower aggression and impulsivity. It can also be used to help reduce GI inflammation resulting from too much serotonin in GI tract.

Thus, for example, in some embodiments, there is provided a method of increasing brain serotonin level in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 TU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin level and decreasing peripheral serotonin level in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of increasing brain serotonin level and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin level and decreasing peripheral serotonin level in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 TU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of increasing the relative ratio of the brain serotonin level to the peripheral serotonin levels in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of increasing the relative ratio of the brain serotonin level to the peripheral serotonin levels in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

The methods described herein may also lead to increase in oxytocin levels. Oxytocin is a neuropeptide hormone that is formed from the precursor protein oxytocin/meurophysin I prepropeptide (OXT) and acts on oxytocin receptors (OXTR), which are distributed throughout the limbic system of the brain and in breast and placental tissues. Oxytocin is important for both aspects of socialization including social comfort and social pain and it works together with serotonin to reward social interactions, suggesting that it may be important for reinforcing correct social behavior. We found that both OXT and OXTR contain putative VDREs which mostly appear to be consistent with transcriptional activation, suggesting that vitamin D could regulate both the production of the oxytocin hormone and the response to it. This suggests that vitamin D could modulate oxytocin synthesis as well as the response to the neuropeptide itself in different tissues, with important implications for benefiting social behavior. The methods described herein thus may also lead to any one of more benefits oxytocin provides.

Thus, in some embodiments, there is provided a method of increasing brain serotonin and oxytocin levels in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin and oxytocin levels and decreasing peripheral serotonin level in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of increasing brain serotonin and oxytocin levels in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin and oxytocin levels and decreasing peripheral serotonin level in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

The methods described herein may also lead to increase in vasopressin signaling, for example by modulating vasopressin receptor levels. Vasopressin is a neuropeptide hormone that regulates many different social and emotional behaviors including social recognition, social bonding, exploration, anxiety, and aggression. The social behavioral effects of vasopressin are mainly mediated through the arginine vasopressin receptor 1A (AVPR1A) and are more pronounced in males, which have a higher expression level of AVPR1A receptors. The AVPR1A gene has been identified as an autism susceptibility gene and microsatellite variants have been linked to autism. Furthermore, it has been demonstrated that the common genetic variants of the AVPR1A gene linked to autism result in lower mRNA expression and are associated with hyperactivation of the amygdala, which is known to be connected with the diminished gaze fixation in autistic individuals. We hypothesize that common variants of the AVPR1A gene that have been linked to autism have decreased transcriptional activity leading to higher amygdala activation and decreased eye gaze in autistic individuals, and that vitamin D is important for normal expression of AVPR1A and, thus, the vasopressin receptor, which may be critical during early brain development. The methods described herein thus may also lead to any one of more benefits vasopressin provides.

Thus, in some embodiments, there is provided a method of increasing brain serotonin and vasopressin receptor levels in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin and vasopressin receptor levels and decreasing peripheral serotonin level in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of increasing brain serotonin and vasopressin receptor levels in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing brain serotonin and vasopressin receptor levels and decreasing peripheral serotonin level in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 TU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

Aberrant serotonin signaling has been identified as a common denominator in neurodevelopmental and neuropsychiatric disorders including autistic spectrum disorder spectrum disorder, attention deficit, hyperactivity disorder, schizophrenia, bipolar disorder, and anti-social behavior disorders. However, up until now no causal mechanism for the underlying serotonergic dysfunction has been established. The present invention provides one consistent pathological explanation that is universal to these neurodevelopmental and neuropsychiatric disorders: high levels of serotonin in peripheral blood and low levels of serotonin in the brain. The methods described herein are therefore useful for treating any one of these disorders. Further, the methods described herein are useful for improving pro-social behavior and cognitive function in an individual, including a healthy individual.

Thus, in some embodiments, there is provided a method of treating (or preventing, delaying the onset of, or alleviating one or more symptoms of) a brain dysfunction disorder in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of treating (or preventing, delaying the onset of, or alleviating one or more symptoms of) a brain dysfunction disorder in an individual and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

“Brain dysfunction disorder” discussed herein includes, but is not limited to, autistic spectrum disorder, defective cognitive function, dementia, mood disorder, attention deficit hyperactivity disorder, schizophrenia, bipolar disorder, and anti-social behavior disorders, impulse behavior disorders.

In some embodiments, there is provided a method of preventing autistic spectrum disorder (including delaying onset of autistic spectrum disorder) in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of preventing autistic spectrum disorder (including delaying onset of autistic spectrum disorder) in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of treating autistic spectrum disorder in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of treating autistic spectrum disorder in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of alleviating at least one symptom of autistic spectrum disorder in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of alleviating at least one symptom of autistic spectrum disorder in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

Symptoms of autistic spectrum disorder described herein include, but are not limited to, any one or more stereotypical behaviors associated with autistic spectrum disorder, such as lack of social interaction, diminished ability to interpret emotional facial expressions, repetitive behavior, social anxiety, tantrums, and aggressive outbursts including self-injurious behavior.

In some embodiments, there is provided a method of treating (or preventing, delaying onset of, or alleviating at least one symptom of) a mood disorder in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 TU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, there is provided a method of treating (or preventing, delaying onset of, or alleviating at least one symptom of) a mood disorder in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

Mood disorders include, but is not limited to, depressive disorders, bipolar disorders, substance induced mood disorders (such as alcohol induced mood disorders, benzodiazepine induced mood disorders, and interferon-alpha induced mood disorders). Depressive disorders include major depressive disorder (MDD, commonly called major depression, unipolar depression, or clinical depression), dysthymia, and depressive disorder not otherwise specified (DD-NOS). Also included herein are atypical depression, melancholic depression, psychotic major depression (PMD, also known as psychotic depression), catatonic depression, postpartum depression (PPD), and seasonal affective disorder (SAD).

In some embodiments, there is provided a method of treating (or preventing, delaying onset of, or alleviating at least one symptom of) a psychiatric disorder in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, there is provided a method of treating (or preventing, delaying onset of, or alleviating at least one symptom of) a psychiatric disorder in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

Symptoms of psychiatric disorders include, but are not limited to, abnormal behavior, abnormal dreams, aggression, agitation, anger, anxiety, impulsive behavior, apathy, confusional state, delusion, depression, hallucination, vial hallucination, insomnia, increased libido, mood alternation, mood swing, lack of focus and attention, hyperactivity, anti-social behavior, nightmare, paranoia, psychotic disorder, and sleep disorder.

Psychiatric disorders described herein include, but are not limited to, Acute Stress Disorder, Adjustment Disorder, Adjustment Disorder with Anxiety, Adjustment Disorder with Depressed Mood, Adjustment Disorder with Disturbance of Conduct, Adjustment Disorder with Mixed Anxiety and Depressed Mood, Adjustment Disorder with Mixed Disturbance of Emotions and Conduct, Agoraphobia without History of Panic Disorder Anxiety Disorders, Anorexia Nervosa Eating Disorders, Antisocial Personality Disorder, Personality Disorders, Anxiety Disorder Due to Medical Condition, Anxiety Disorder NOS, Avoidant Personality Disorder, Bipolar Disorder NOS, Bipolar I Disorder Most Recent Episode Depressed (in full remission), Bipolar I Disorder Most Recent Episode Depressed (in partial remission), Bipolar I Disorder most recent episode depressed (mild), Bipolar I Disorder Most Recent Episode Depressed (Moderate), Bipolar I Disorder most recent episode depressed (severe with psychotic features), Bipolar I Disorder, most recent episode depressed (severe without psychotic features), Bipolar I Disorder most recent episode depressed (unspecified), Bipolar I Disorder most recent episode manic (in full remission), Bipolar I Disorder most recent episode manic (in partial remission), Bipolar I Disorder most recent episode manic (mild), Bipolar I Disorder most recent episode manic (moderate), Bipolar I Disorder most recent episode manic (severe with psychotic features), Bipolar I Disorder most recent episode manic (severe without psychotic features), Bipolar I Disorder most recent episode manic (unspecified), Bipolar I Disorder most recent episode mixed (in full remission), Bipolar I Disorder, most recent episode mixed in partial remission, Bipolar I Disorder most recent episode mixed (mild), Bipolar I Disorder most recent episode mixed (moderate), Bipolar I Disorder most recent episode mixed (severe with psychotic features), Bipolar I Disorder most recent episode mixed severe without psychotic features, Bipolar I Disorder most recent episode mixed (unspecified), Bipolar I Disorder most recent episode unspecified, Bipolar I Disorder most recent episode hypomanic, Bipolar I Disorder single manic episode in full remission, Bipolar I Disorder single manic episode in partial remission, Bipolar I Disorder single manic episode (mild), Bipolar I Disorder single manic episode (moderate), Bipolar I Disorder single manic episode (severe with psychotic features), Bipolar I Disorder single manic episode severe without psychotic features, Bipolar I Disorder single manic episode (unspecified), Bipolar II Disorder, Body Dysmorphic Disorder, Borderline Personality Disorder, Breathing-Related Sleep Disorder, Brief Psychotic Disorder, Bulimia Nervosa, Circadian Rhythm Sleep Disorder, Conversion Disorder, Cyclothymic Disorder, Delusional Disorder, Dependent personality Disorder, Depersonalization disorder, Depressive Disorder NOS, Dissociative Amnesia, Dissociative Disorder NOS, Dissociative fugue, Dissociative Identity Disorder, Dissociative Disorders, Dyspareunia, Dyssomnia NOS, Dyssomnia Related to (Another Disorder), Dysthymic Disorder, Eating disorder NOS, Exhibitionism, Female Dyspareunia Due to Medical Condition, Female Hypoactive Sexual Desire Disorder Due to Medical Condition, Female Orgasmic Disorder, Female Sexual Arousal Disorder, Fetishism Sexual Disorders, Frotteurism Sexual Disorders, Gender Identity Disorder in Adolescents or Adults, Gender Identity Disorder in Children, Gender Identity Disorder NOS, Generalized Anxiety Disorder, Histrionic Personality Disorder, Hypoactive Sexual Desire Disorder, Hypochondriasis, Impulse—Control Disorder NOS, Insomnia Related to Another Disorder, Intermittent Explosive Disorder, Kleptomania, Major Depressive Disorder Recurrent (in full remission), Major Depressive Disorder Recurrent (in partial remission), Major Depressive Disorder Recurrent (Mild), Major Depressive Disorder recurrent (moderate), Major Depressive Disorder recurrent (severe with psychotic features), Major Depressive Disorder, Recurrent (severe without psychotic features), Major Depressive Disorder Recurrent (unspecified), Major Depressive Disorder Single Episode (in full remission), Major Depressive Disorder single episode (in partial remission), Major Depressive Disorder single episode (Mild), Major Depressive Disorder single episode, Major Depressive Disorder single episode (severe with psychotic features), Major Depressive Disorder single episode (severe without psychotic features), Major Depressive Disorder single episode (unspecified), Male Dyspareunia Due to Medical Condition, Male Erectile Disorder, Male Erectile Disorder Due to Medical Condition, Male Hypoactive Sexual Desire Disorder Due to Medical Condition, Male Orgasmic Disorder, Mood Disorder Due to Medical Condition, Narcissistic Personality Disorder, Narcolepsy Sleep Disorders, Nightmare Disorder Sleep Disorders, Obsessive Compulsive Disorder, Obsessive-Compulsive Personality Disorder, Other Female Sexual Dysfunction Due to Medical Condition, Other Male Sexual Dysfunction Due to Medical Condition, Pain Disorder Associated with both Psychological Factors and Medical Conditions, Pain Disorder Associated with Psychological Features, Panic Disorder with Agoraphobia, Panic Disorder without Agoraphobia, Paranoid Personality Disorder, Paraphilia NOS, Parasomnia NOS, Pathological Gambling, Pedophilia Sexual Disorders, Personality Disorder NOS, Posttraumatic Stress Disorder, Premature Ejaculation, Primary Hypersomnia, Primary Insomnia, Psychotic Disorder Due to Medical Condition with Delusions, Psychotic Disorder Due to Medical Condition with Hallucinations, Psychotic Disorder NOS, Pyromania, Schizoaffective Disorder, Schizoid Personality Disorder, Schizophrenia Catatonic Type, Schizophrenia Disorganized Type, Schizophrenia Paranoid Type, Schizophrenia Residual Type, Schizophrenia Undifferentiated Type, Schizophreniform Disorder, Schizotypal Personality Disorder, Sexual Aversion Disorder, Sexual Disorder NOS, Sexual Dysfunction NOS, Sexual Masochism, Sexual Sadism, Shared Psychotic Disorder, Sleep Disorder Due to A Medical Condition Hypersomnia Type, Sleep Disorder Due to A Medical Condition Insomnia Type, Sleep Disorder Due to A Medical Condition Mixed Type, Sleep Disorder Due to A Medical Condition Parasomnia Type, Sleep Terror Disorder, Sleepwalking Disorder, Social Phobia, Somatization Disorder, Somatoform Disorder NOS, Specific Phobia, Trichotillomania, Undifferentiated Somatoform Disorder, Vaginismus Sexual Disorders, and Voyeurism, compulsive gambling disorder, and attention deficit hyperactivity disorder.

The methods described herein are also useful for improving pro-social behavior and/or cognitive function in individuals, including healthy individuals.

“Pro-social behavior” used herein includes, but is not limited to, less quarrelsome, more agreeable, less aggressive, less impulsive, more empathetic and ability to interpret others emotions/actions, more altruistic, less selfish, and better social skills. Improvement of pros-social behavior thus includes improvement on any one or more of the pro-social behaviors described herein.

“Cognitive function” used herein includes, but is not limited to, better executive function, better episodic memory, semantic memory, improved learning, better long-term planning and foresight, less impulsivity, better attention, and focus. Improvement of cognitive function thus includes improvement on any one or more of the cognitive functions described herein.

Thus, in some embodiments, there is provided a method of improving pro-social behavior in an individual (such as a healthy individual), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of improving pro-social behavior in an individual (such as a healthy individual) and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of improving cognitive function in an individual (such as a healthy individual), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of improving cognitive function in an individual (such as a healthy individual) and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of improving pro-social behavior and cognitive function in an individual (such as a healthy individual), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of improving pro-social behavior and cognitive function in an individual (such as a healthy individual) and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of modulating satiety in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of modulating satiety in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of treating (or preventing) obesity in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of treating (or preventing) obesity in an individual and concomitantly reducing inflammation in the GI tract, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 TU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, the individual is a healthy individual. In some embodiments, the individual shows one or more symptoms of autistic spectrum disorder, mood disorder, psychiatric disorder, cognitive dysfunction, attention deficit hyperactivity disorder, schizophrenia, bipolar disorder, anti-social behavior disorders, impulsive behavior disorders, aggressive behavior disorders, or depression. In some embodiments, the individual shows one of more signs of impairment of cognitive functions. In some embodiments, the individual is diagnosed with autistic spectrum disorder, mood disorder, psychiatric disorder, cognitive dysfunction, attention deficit hyperactivity disorder, schizophrenia, bipolar disorder, anti-social behavior disorders, impulsive behavior disorders, aggressive behavior disorders, or depression. In some embodiments, the individual is diagnosed or genetically prone to one or more risks of developing one or more diseases described herein. In some embodiments, the individual has one or more risk factors associated with one or more diseases discussed herein.

While most of the TPH2-generated serotonin is found inside the blood-brain barrier, there are some neurons found in the GI tract (enteric neurons), which also express TPH2. Serotonin produced from TPH2-expressing enteric neurons is required for gut motility, whereas serotonin generated from TPH1-expressing gut enterochromaffin cells regulate inflammation. We propose that GI inflammation (for example that observed in autistic individuals) may be a direct result of elevated serotonin in the GI tract due to increased TPH1 expression in the presence of low vitamin D levels. In addition, we predict that vitamin D and tryptophan supplementation will also increase enteric-derived serotonin (via increasing TPH2 expression), thereby improving gut motility and constipation. Simultaneously, vitamin D should help lower GI inflammation by decreasing the serotonin level in the enterochromaffin GI cells through transcription repression of TPH1. Therefore, vitamin D and tryptophan supplementation would increase TPH2 and decrease TPH1 expression, because it contains a VDRE consistent with transcriptional activation and repression, respectively. This would result in normalizing serotonin levels in the gut and concomitantly increasing gut motility while reducing GI inflammation and irritation.

Thus, in some embodiments, there is provided a method of increasing gut motility in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of increasing gut motility and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

In some embodiments, there is provided a method of reducing constipation in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of reducing constipation and concomitantly reducing inflammation in the gastrointestinal tract (GI tract), comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, the vitamin D and the tryptophan are administered separately. In some embodiments, the vitamin D and the tryptophan are administered simultaneously. In some embodiments, the vitamin D and the tryptophan are administered in a single composition (such as the vitamin D/tryptophan composition described herein). In some embodiments, the method further comprises administering to the individual one or more of: vitamin B6, BH4 (tetrahydrobiopterin), omega-3 long chain polyunsaturated fatty acid (LCPUFA), and iron. In some embodiments, the amount of the LCPUFA is at least about 500 mg per day, including for example about 500 to about 6000 mg, about 1000 to about 6000 mg, about 2000 to about 6000 mg, about 3000 to about 5000 mg, or about 3000 to about 4000 mg per day.

Serotonin is known to increase angiogenesis in endothelial cells and stimulates the growth, proliferation and migration of blood vessels. Qin et al., Blood 121, 2154-2164 (2013). We hypothesize that serotonin's role in angiogenesis extends to inside the blood brain barrier as well where it acts as a neuroangiogenic factor, promoting the growth of new blood vessels and neurogenesism and that TPH2-produced serotonin plays an important role in neurogenesis possibly through angiogenesis. We propose that TPH2-generated serotonin in the brain could promote the growth of new blood vessels and angiogenesis; which may have important implications for adult neurogenesis in neurodegenerative diseases such as Alzheimer's and Parkinson's well as for stoke and ischemic brain damage. Supplementation with vitamin D and tryptophan as described herein can be used to increase neurogenesis in normal and diseased individuals. In normal people, vitamin D and tryptophan can boost neurogenesis and angiogenesis in the brain. Furthermore, vitamin D and tryptophan may help prevent neurodegenerative diseases and stroke.

Thus, in some embodiments, there is provided a method of increasing neurogenesis and angiogenesis in the brain of an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of reducing risk of neurodegenerative disease in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day. In some embodiments, there is provided a method of reducing risk of stroke in an individual, comprising administering (such as orally administering) to the individual an effective amount of vitamin D and an effective amount of tryptophan, wherein the amount of the vitamin D is at least about 500 IU (such as at least about any of 600, 700, 800, 900, 1000, 1500, 2000, 2500, 3000, 3500, or 4000 IU) per day, and the amount of the tryptophan is at least about 100 mg (such as at least about any of 200, 500, 800, 1000, 2000, 3000, 4000, 5000, or 6000 mg) per day.

In some embodiments, the individual has a high level of melanin. In some embodiments, the individual has a low level of melanin. In some embodiments, the individual has dark skin. In some embodiments, the individual has an African black ethnicity.

In some embodiments, the individual is an obese individual. In some embodiments, the individual is an overweight individual. In some embodiments, the obese individual has a body mass index (BMI) greater than about 30, 35, 40, or 45. In some embodiments, the obese individual has a BMI greater than about 60 percentile (such as any of 65, 70, 75, 80, 85, 90, or 95 percentile) of all people in the individual's age group.

In some embodiments, the individual is a human who is younger than about 60 years old (including for example an individual younger than about any of 50, 40, 30, 25, 20, 15, 14, 13, 12, 11, 10, 9, 8, 7, 6, 5, 4, 3, or 2 years old). In some embodiments, the individual is a human who is older than about 50 years old (including for example an individual older than about any of 55, 60, 65, 70, 75, or 80 years old). In some embodiments, the individual is a man. In some embodiments, the individual is a woman. In some embodiments, the individual is a pregnant individual, and the methods are useful for preventing the pregnant individual from having a child that has a brain dysfunction disorder (such as any of the disorders described herein).

In some embodiments, the individual has a mood disorder, such as any of the mood disorder described herein. In some embodiments, the individual has a psychiatric disorder, such as any of the psychiatric disorder described herein. In some embodiments, the individual has autistic spectrum disorder, including severe autistic spectrum disorder or mild-to-moderate autistic spectrum disorder. In some embodiments, the individual has autistic spectrum disorder with an ASD severity of 15-29, 30-36, or 37-60 based on the Childhood Autistic spectrum disorder Rating Scale total score.

In some embodiments, the individual has a high peripheral level of TPH1. In some embodiments, the individual is selected for treatment based on high peripheral level of TPH1. Methods of determining peripheral TPH1 levels discussed in the section below are applicable to any one of the methods described herein.

The various behavioral and cognitive functions described herein can be assessed at the baseline and after treatment, for example 4, 8, and 16 weeks after treatment. The MATRICS consensus cognitive battery (MCCB) can be used, for example, to assess cognitive functioning. Miller et al., Journal of Psychopathology and Behavioral Assessment 32, 323-332 (2010). The MCCB includes speed of processing, attention/vigilance, working memory, verbal memory, visual memory, reasoning and problem solving, and social cognition. Miller et al. The well-established Barratt Impulsiveness Scale (BIS-11) self-report questionnaire can be used to determine impulsivity. Patton et al., Journal of Clinical Psychology 51, 768-774 (1995); Stanford et al., Personal Indiv Diff 47, 385-395. (2009). The measure includes 30 items scored on a 4-point scale (l=never, 4=always) describing common impulsive or non-impulsive behaviors and preferences. The dependent measures are summed scores for each of the scales. Sensory gating can be assessed by the PPI and P50 suppression tests as previously described. Miller et al.

Cognitive function including language skills can be assessed, for example, by the PLS method described in Zimmerman I L, S. V., Pond R E. Preschool Language Scale Fourth Edition Harcourt Assessment: San Antonio, Tex., USA, 2002. (2002). The PLS consists of receptive, expressive and total communication scores. Adaptive behavior including receptive, expressive and writing communication, personal, domestic and community daily living skills, interpersonal relations, play and coping skills will be assessed using the VABS. Sparrow S, C. D., Balla D. ineland Adaptive Behavior Scales 2nd edn. Pearson Assessments: Bloomington, Minn., USA, 2005. (2005). Social skills will be assessed using a observational parent-child interaction scale, and parent and teacher SRS. JN., C. The Social Responsiveness Scale. Western Psychological Services: Los Angeles, Calif., USA, 2002. (2002). Assessments include, but are not limited to, responsiveness, social engagement and affect as previously reported with higher scores on the observation scales representing better performance. Bolte et al., Journal of Autistic Spectrum Disorder and Developmental Disorders 41, 66-72 (2011). Behavior can be assessed using the parent and teacher Aberrant Behavior Checklist (ABC). Aman et al., American Journal of Mental Deficiency 89, 485-491 (1985). Subscales represent irritability, social withdrawal, stereotypic behaviors, hyperactivity and inappropriate speech. Autistic spectrum disorder symptoms can be assessed using the autistic spectrum disorder symptoms questionnaire (ASQ), a DSM-V-TR-based checklist developed by The Center for Autistic spectrum disorder and Related Disorders (Tarzana, Calif., USA). American Psychiatric Association Diagnostic and Statistical Manual of Mental Disorders. 5th ed. (2013).

### Vitamin D/Tryptophan Compositions

The present application in another aspect provides a composition comprising vitamin D and tryptophan. In some embodiments, the composition is a nutraceutical composition. In some embodiments, the composition is a pharmaceutical composition, for example the composition may further comprise one or more drugs (e.g., one or more active ingredients) suitable for any one or more uses described herein. In some embodiments, the pharmaceutical composition further comprises a pharmaceutically acceptable carrier. In some embodiments, the composition is substantially free of other active ingredients.

Vitamin D described herein includes vitamin D2 (ergocalciferol), vitamin D3 (cholecalciferol), vitamin D4, vitamin D5, vitamin D6, and vitamin D7. The term vitamin D also includes metabolites and other analogues of these substances. The term “vitamin D” also encompasses various ester forms of vitamin, including, but not limited to, vitamin D acetate, vitamin D propionate, vitamin D caproate, vitamin D caprate, vitamin D laurate, vitamin D myristate, vitamin D palmitate, vitamin D stearate, vitamin D oleate, vitamin D linolenate, vitamin D arachidonate, vitamin D linoleate, vitamin D eicosapentaenoate, vitamin D docosahexaenoate, vitamin D benzoate, vitamin D lactate, vitamin D sorbate, vitamin D glycinate (alpha-amino acetate), vitamin D alanate (alpha-amino propionate), vitamin D succinate, vitamin D fumarate, vitamin D polyethylene glycol succinate, or a mixture thereof.

In some embodiments, the composition comprises vitamin D in the amount of about 100% to about 400% RDA, including for example about 100% to about 200% RDA. In some embodiments, the composition comprises about 100 to about 4000 IU, including for example about 500 to about 4000 IU, about 500 to about 3000 IU, about 500 IU to about 2000 IU, or about 1000 IU. In some embodiments, the vitamin D is at least about 1%, 2%, 3%, 4%, 5%, 10%, or 15% (w/w) of the composition, including any percentages in between these values.

In some embodiments, the composition comprises tryptophan in the amount of about 100% to about 400% RDA, including for example about 100% to about 200% RDA. In some embodiments, the composition comprises about 0.1 to about 6 grams of tryptophan, including for example about any of 0.2-0.3, 0.3-0.4, 0.4-0.5, 0.5-0.6, 0.6-0.7, 0.7-0.8, 0.8-0.9, 0.9-1.0, 1.0-1.5, 1.5-2.0, 2.0-2.5, 2.5-3.0, 3.0-3.5, 3.5-4.0, 4.0-4.5, 4.5-5.0, 5.0-5.5, or 5.5-6.0 grams. In some embodiments, the composition comprises at least about any of 0.1, 0.2, 0.3, 0.4, 0.5, 0.6, 0.7, 0.8, 0.9, 1.0, 1.2, 1.4, 1.6, 1.8, 2.0, 2.5, 3, 3.5, 4.0, 4.5, 5.0, 5.5, or 6.0 grams. In some embodiments, the tryptophan is at least about 10%, 20%, 30%, 40%, 50%, 60%, 70%, 80%, or 90% (w/w) of the composition, including any percentages in between these values.

In some embodiments, there is provided a composition comprising vitamin and amino acid, wherein at least about 5% (including for example at least about any of 6%, 7%, 8%, 9%, 10%, or 15%) of the vitamin in the composition is vitamin D. In some embodiments, there is provided a composition comprising vitamin and amino acid, wherein at least about 30% (including for example at least about any of 40%, 50%, 60%, 70%, 80%, 90%, 95%, or 99%) of the amino acid in the composition is tryptophan. In some embodiments, there is provided a composition comprising vitamin and amino acid, wherein at least about 5% (including for example at least about any of 6%, 7%, 8%, 9%, 10%, or 15%) of the vitamin in the composition is vitamin D, wherein at least about 30% (including for example at least about any of 40%, 50%, 60%, 70%, 80%, 90%, 95%, or 99%) of the amino acid in the composition is tryptophan. In some embodiments, the composition further comprises one or more of: vitamin B6, BH4, omega-3 long chain polyunsaturated fatty acid (LCPUFA), and iron. In some embodiments, the composition is essentially free of vitamins other than vitamin D and vitamin B6. In some embodiments, the composition is essentially free of amino acids other than tryptophan.

In some embodiments, there is provided a composition comprising vitamin D and tryptophan, wherein the weight ratio of the vitamin D and the tryptophan in the composition is about 500 IU vitamin D per gram of tryptophan to about 3000 IU vitamin D per gram of tryptophan, including for example about 1000 IU vitamin D per gram of tryptophan to about 2000 IU vitamin D per gram of tryptophan, or about 4000 IU vitamin D per 3 grams of tryptophan. In some embodiments, the composition further comprises one or more of: vitamin B6, BH4, omega-3 long chain polyunsaturated fatty acid (LCPUFA), and iron. In some embodiments, the weight ratio of LCPUFA and tryptophan in the composition is about any of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10.

In some embodiments, there is provided a composition comprising vitamin D and tryptophan, wherein the amount of the vitamin D in the composition is at least about 500 IU (such as at least about any of 600, 700, 800, 900, 1000, 1500, 2000, 2500, 3000, 3500, or 4000 IU), and the amount of the tryptophan in the composition is at least about 100 mg (such as at least about any of 200, 500, 800, 1000, 2000, 3000, 4000, 5000, or 6000 mg). In some embodiments, the composition further comprises one or more of: vitamin B6, BH4, omega-3 long chain polyunsaturated fatty acid (LCPUFA), and iron. In some embodiments, the amount of the LCPUFA is at least about 500 mg, including for example about 500 to about 6000 mg, about 1000 to about 6000 mg, about 2000 to about 6000 mg, about 3000 to about 5000 mg, or about 3000 to about 4000 mg.

In some embodiments, there is provided a unit dosage form comprising vitamin D and tryptophan, wherein the amount of the vitamin D in the composition is at least about 500 IU (such as at least about any of 600, 700, 800, 900, 1000, 1500, 2000, 2500, 3000, 3500, or 4000 IU), and the amount of the tryptophan in the composition is at least about 100 mg (such as at least about any of 200, 500, 800, 1000, 2000, 3000, 4000, 5000, or 6000 mg). In some embodiments, the unit dosage form further comprises one or more of: vitamin B6, BH4, omega-3 long chain polyunsaturated fatty acid (LCPUFA), and iron. In some embodiments, the amount of the LCPUFA is at least about 500 mg, including for example about 500 to about 6000 mg, about 1000 to about 6000 mg, about 2000 to about 6000 mg, about 3000 to about 5000 mg, or about 3000 to about 4000 mg. “Unit dose” used herein refers to the amount per serving size of the composition. In some embodiments, the unit dose is the amount per daily serving of the composition, but in some embodiments, one daily serving may contain multiple unit doses.

The unit dosage forms can be provided in the form of tablets, capsules, packaged powder, etc. In some embodiments, one unit dosage form contains the total daily amount of the vitamin D and tryptophan. In some embodiments, the daily amount of the vitamin D and tryptophan is contained in more than one unit dosage forms, for example in two, three, four, or more unit dosage forms.

In some embodiments, the compositions described herein comprise vitamin B6. Vitamin B6 is a water soluble vitamin that was first isolated in 1930's. There are six forms of vitamin B6: pyridoxal, pyridoxine, pyridoxamine, and their active phosphate derivatives including pyridoxal 5′-phosphate (PLP) and pridoxamine 5′-phosphate and also the inactive derivative pyridoxine 5′-phosphate. In some embodiments, the composition comprises vitamin B6 in the amount of about 50% to about 200%, including for example about 100% RDA. In some embodiments, the composition comprises about 0.6 to about 2.5 mg, including for example about 1.3 mg vitamin B6. In some embodiments, the composition comprises about 0.001% to about 0.005%, including for example about 0.002% to about 0.0025% vitamin B6.

In some embodiments, the composition described herein further comprises BH4. BH4 (tetrahydrobiopterin) is a naturally occurring essential cofactor of the three aromatic amino acid hydroxylase enzymes, used in the degradation of amino acid phenylalanine and in the biosynthesis of serotonin, melatonin, dopamine, norepinephrine (noradrenaline), epinephrine (adrenaline), and is a cofactor for the production of nitric oxide (NO) by the nitric oxide synthases. In some embodiments, the composition comprises BH4 in the amount of about 30% to about 150%, including for example about 50% to about 100%, or 66% RDA. In some embodiments, the composition comprises about 0.75 to about 3.5 μg, including for example about 1 to about 2 μg, or 1.6 μg BH4. In some embodiments, the composition comprises about 0.0000015% to about 0.000005%, including for example about 0.0000027% BH4. In some embodiments, the composition comprises at least about 0.5 mg BH4, including for example at least about 1 mg, 1.5 mg, 2 mg BH4. This is especially useful for elderly individuals, a large percent of who do not readily absorb ingested BH4.

In some embodiments, the composition further comprises iron. Iron can be provided by iron citrate, ferritin iron, EDTA iron, ferric sodium pyrophosphase, and/or iron fumarate. In some embodiments, the iron is provided in the form(s) of iron chelates. Suitable iron chelates include, but are not limited to, EDTA iron chelate, DPTA iron chelate, and iron citrate. In some embodiments, the iron is provided in a natural form, for example, in the form of a protein complex naturally exist in the human body. In some embodiments, the composition comprises iron in the amount of about 10% to about 100%, including for example about 20% to about 80%, such as 50% RDA. In some embodiments, the composition comprises about 2 to about 10 mg, including for example about 4 mg iron. In some embodiments, the composition comprises about 0.002% to about 0.01%, including for example about 0.007% iron.

In some embodiments, the composition further comprises an omega-3 long chain polyunsaturated fatty acid (LCPUFA). In some embodiments, the amount of the LCPUFA in the composition is at least about 500 mg, including for example about 500 to about 6000 mg, about 1000 to about 6000 mg, about 2000 to about 6000 mg, about 3000 to about 5000 mg, or about 3000 to about 4000 mg. LCPUFA is important in brain development and long term health. Omega-3 LCPUFA, which is found primarily in fish oils, can also be synthesized in the body from alpha linolenic acid (ALA) found in nuts, and some vegetable and animal fats. However, the rate of synthesis of EPA and DHA from ALA may not be sufficient for optimal function. LCPUFA described herein include fish oil, eicosapentaenoic acid (EPA), blue algae omega-3, docosahexaenoic acid (DHA), and linolenic acid. In some embodiments, the LCPUFA is algae-derived DHA. In some embodiments, the composition comprises both omega-3 and omega-6 fatty acids. In some embodiments, the ratio of omega-3 and omega-6 fatty acids in the composition is less than about 5:1, including for example less than about 4:1, 3:1, 2:1, 1.6:1, or 1:1. In some embodiments, the composition comprises about 500 to about 6000 mg, about 1000 to about 6000 mg, about 2000 to about 6000 mg, about 3000 to about 5000 mg, or about 3000 to about 4000 mg DHA. In some embodiments, the composition comprises about 0.1% to about 1%, including for example about 0.6% to about 0.7% DHA.

The compositions described herein may be formulated and administered in any known or otherwise suitable oral product form. Any solid, liquid, or powder form, including combinations or variations thereof, are suitable for use herein, provided that such forms allow for safe and effective oral delivery to the individual of the essential ingredients as also defined herein.

The compositions described herein may also be formulated in product forms such as capsules, tablets, pills, caplets, gels, liquids (e.g., suspensions, solutions, emulsions), powders or other particulates, and so forth. These product forms preferably contain only the essential ingredients as described herein, optionally in combination with other actives, processing aids or other dosage form excipients.

In some embodiments, the composition is provided in the form of nutrition bars. The nutrition bar in some embodiments is provided in a sealed package, for example a package scaled under nitrogen. In some embodiments, one or more nutrition bars are contained in a single sealed package. In some embodiments, the composition is provided in the form of nutritional bites (e.g., a plurality of smaller dietary product dosage forms in a single package). In some embodiments, the composition is provided in the form of nutritional sticks.

Solid dosage forms for oral administration (e.g., capsules, tablets, pills, powders, granules, and the like) may be prepared, e.g, by mixing the active ingredient(s) with one or more pharmaceutically acceptable carrier and, optionally, one or more fillers, extenders, binders, humectants, disintegrating agents, solution retarding agents, absorption accelerators, wetting agents, absorbents, lubricants, and/or coloring agents. Solid compositions of similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may be prepared with coatings and shells, such as enteric coatings and other coatings well known in the art. The formulations may be sterilized by, for example, filtration through a bacterial-retaining filer. These composition may also contain opacifying agent. In some embodiments, the composition is in microencapsulated form.

The compositions described herein in some embodiments can be formulated as liquids, such as milk-based liquids, soy-based liquids, low-pH liquids, and liquid reconstituted from powders. In some embodiments, the compositions are provided in the form of semi-liquids, such as yogurt. In some embodiments, the composition is provided in the form of kefir or sprinkles. Exemplary liquid dosage forms for oral administration include emulsions, microemulsions, solutions, suspensions, syrups and elixirs. The liquid dosage forms may contain suitable insert diluents commonly used in the art. In some embodiments, the liquid dosage forms may further include wetting agents, emulsifying and suspending agents, sweetening, flavoring, coloring, perfuming and preservative agents.

The compositions described herein may also include a variety of different product forms, including any conventional or otherwise known food product form, some non-limiting examples of which include confectionery products, cereals, food condiments (e.g., spreads, powders, sauces, jams, jelly, coffee creamer or sweetener), baking or cooking materials (e.g., flour, fats or oils, butter or margarine, breading or baking mixes), salted or seasoned snacks (e.g., extruded, baked, fried), beverages (e.g., coffee, juice, carbonated beverage, non-carbonated beverage, tea, ice-cream based drinks), snack or meal replacement bars, smoothies, breakfast cereals, cheeses, gummy products, salted or unsalted crisp snacks (e.g., chips, crackers, pretzels), dips, baked goods (e.g., cookies, cakes, pies, pastries, bread, bagels, croutons, dressings, dry mixes (e.g., mixes for muffins, cookies, waffles, pancakes, beverages), frozen desserts (e.g., ice cream, fudge bars, frozen yogurt), pudding, flavored or unflavored gelatin, refrigerated dough (e.g., cookies, bread, brownies), milk or soy-based smoothies, yogurt or yogurt-based drinks, frozen yogurt, soy milk, soups, and snacks.

The compositions described herein may also be formulated for parenteral (such as intravenous) administration.

### Methods Based on Peripheral TPH1 Levels in an Individual

In another aspect, the present application provides diagnosis and treatment methods based on TPH1 levels. The methods described herein are based on the finding that vitamin D differentially regulates TPH2 in the brain and TPH1 in the peripheral tissue. It is thus contemplated that the peripheral level of TPH1 would serve as an accurate and specific indicator of one or more of the following in an individual: 1) low vitamin D status; 2) low serotonin in the brain; 3) risk for a disorder associated with low brain serotonin level (such as any one of the disorders discussed above); and 4) risk of developing inflammation in the GI tract (such as leaky gut syndrome).

Thus, in some embodiments, there is provided a method of assessing the need for vitamin D supplementation in an individual, comprising determining the peripheral TPH1 level in the individual, wherein a high TPH1 level is indicative that the individual is in need of vitamin D supplementation. In some embodiments, there is provided a method of assessing the need for vitamin D supplementation in an individual, comprising determining the peripheral TPH1 level in the individual using an antibody recognizing TPH1, wherein a high TPH1 level is indicative that the individual is in need of vitamin D supplementation. In some embodiments, the TPH1 level is determined by ELISA (enzyme-linked immunosorbant assay). In some embodiments, the method further comprises administering vitamin D to the individual. In some embodiments, the method further comprises administering vitamin D and tryptophan in this individual, for example according to any of the methods described herein that comprise administration of vitamin D and tryptophan.

In some embodiments, there is provided a method of assessing the brain serotonin level in an individual, comprising determining the peripheral TPH1 level in the individual, wherein a high TPH1 level is indicative that the individual has a low brain serotonin level. In some embodiments, there is provided a method of assessing the brain serotonin level in an individual, comprising determining the peripheral TPH1 level in the individual using an antibody recognizing TPH1, wherein a high TPH1 level is indicative that the individual has a low brain serotonin level. In some embodiments, the TPH1 level is determined by ELISA. In some embodiments, the method further comprises administering to the individual an agent that increases brain serotonin level (such as vitamin D, tryptophan, or the combination thereof).

In some embodiments, there is provided a method of assessing the risk of developing a disorder associated with a low brain serotonin level in an individual, comprising determining the peripheral TPH1 level in the individual, wherein a high TPH1 level is indicative that the individual has a high risk of developing a disorder associated with a low brain serotonin level. In some embodiments, there is provided a method of assessing the risk of developing a disorder associated with a low brain serotonin level in an individual, comprising determining the peripheral TPH1 level in the individual using an antibody recognizing TPH1, wherein a high TPH1 level is indicative that the individual has a high risk of developing a disorder associated with a low brain serotonin level. In some embodiments, the TPH1 level is determined by ELISA. In some embodiments, the method further comprises administering to the individual an agent that increases brain serotonin level (such as vitamin D, tryptophan, or the combination thereof).

In some embodiments, there is provided a method of assessing the risk of developing a brain dysfunction disorder in an individual, comprising determining the peripheral TPH1 level in the individual, wherein a high TPH1 level is indicative that the individual has a high risk of developing a brain dysfunction disorder. In some embodiments, there is provided a method of assessing the risk of developing a brain dysfunction disorder in an individual, comprising determining the peripheral TPH1 level in the individual using an antibody recognizing TPH1, wherein a high TPH1 level is indicative that the individual has a high risk of developing a brain dysfunction disorder. In some embodiments, the TPH1 level is determined by ELISA. In some embodiments, the method further comprises administering to the individual an agent that increases brain serotonin level (such as vitamin D, tryptophan, or the combination thereof).

In some embodiments, there is provided a method of diagnosing a brain dysfunction disorder in an individual, comprising determining the peripheral TPH1 level in the individual, wherein a high TPH1 level is indicative that the individual has a brain dysfunction disorder. In some embodiments, there is provided a method of diagnosing a brain dysfunction disorder in an individual, comprising determining the peripheral TPH1 level in the individual using an antibody recognizing TPH1, wherein a high TPH1 level is indicative that the individual has a brain dysfunction disorder. In some embodiments, the TPH1 level is determined by ELISA. In some embodiments, the method further comprises administering to the individual an agent that increases brain serotonin level (such as vitamin D, tryptophan, or the combination thereof).

In some embodiments, there is provided a method of assessing the severity of a brain dysfunction disorder in an individual, comprising determining the peripheral TPH1 level in the individual, wherein the level of the peripheral TPH1 correlates with the level of the severity of the brain dysfunction disorder. In some embodiments, there is provided a method of assessing the severity of a brain dysfunction disorder in an individual, comprising determining the peripheral TPH1 level in the individual using an antibody recognizing TPH1, wherein the level of the peripheral TPH1 correlates with the level of the severity of the brain dysfunction disorder. In some embodiments, the TPH1 level is determined by ELISA. In some embodiments, the method further comprises administering to the individual an agent that increases brain serotonin level (such as vitamin D, tryptophan, or the combination thereof).

An “agent that increases brain serotonin level” described herein includes vitamin D, tryptophan, or the combination thereof (such as a composition comprising both vitamin D and tryptophan, for example the compositions described herein). It is to be understood that the term “agent that increases brain serotonin level” also encompasses other agents that are known to increase brain serotonin level. These include, but are not limited to, serotonin, serotonin re-uptake inhibitor, 5-HTP (5-hydroxytryptophan, also known as oxitriptan), fenfluramine hydrochloride, and ergot derivative (e.g., bromocriptine, lisuride, pergolide, and mesulergine), Citalopram, dapoxetine, escitalopram, fluoxetine, fluvoxamine, indalpine, paroxetine, sertraline, and zimelidine.

In some embodiments, there is provided a method of assessing the risk of developing GI inflammation in an individual, comprising determining the peripheral TPH1 level in the individual, wherein a high TPH1 level is indicative that the individual has a high risk of developing GI inflammation. In some embodiments, there is provided a method of assessing the risk of developing GI inflammation in an individual, comprising determining the peripheral TPH1 level in the individual using an antibody recognizing TPH1, wherein a high TPH1 level is indicative that the individual has a high risk of developing GI inflammation. In some embodiments, the TPH1 level is determined by ELISA. In some embodiments, the method further comprises administering to the individual an agent that increases brain serotonin level (such as vitamin D, tryptophan, or the combination thereof).

In some embodiments, the TPH1 level is used for the prediction of bone abnormality in an individual. While it was well-known that vitamin D deficiency increases the rate of bone turnover (osteoclastogenesis), partly mediated through elevated levels of parathyroid hormone (PTH), a negative transcriptional target of vitamin D 1-3, we found that TPH1-mediated serotonin production also induces osteoclast formation and causes bone loss. Consistent with this finding, it was previously shown that mice lacking TPH1 display decreased osteoclastogenesis and increased bone mass. Chabbi-Achengli et al., PNAS 109, 2567-2572 (2012). Furthermore, pharmacological inhibition of TPH1 in mice promotes osteoblast formation and increases bone mass in an osteoporosis mouse model. Yadav et al., Nature Medicine 16, 308-312 (2010). Vitamin D may regulate bone mass by a novel mechanism through TPH1 gene repression, thus decreasing the production of serotonin from the gut enterochromaffin cells and increasing osteoblast formation. Therefore, when vitamin D levels are inadequate, this leads to elevated TPH1 expression and higher serotonin levels which break down bone. The present application therefore also provides methods of using peripheral TPH1 expression for the prediction of bone loss and predisposition to osteoporosis and/or osteomalcia (bone pain). If TPH1 levels are high, then treatment with vitamin D supplementation can be used.

Thus, in some embodiments, there is provided a method of assessing the risk of developing bone abnormality (such as bone loss, osteoporosis, and/or osteomalcia) in an individual, comprising determining the peripheral TPH1 level in the individual, wherein a high TPH1 level is indicative that the individual has a high risk of developing bone abnormality. In some embodiments, there is provided a method of assessing the risk of developing bone abnormality (such as bone loss, osteoporosis, and/or osteomalcia) in an individual, comprising determining the peripheral TPH1 level in the individual using an antibody recognizing TPH1, wherein a high TPH1 level is indicative that the individual has a high risk of developing bone abnormality. In some embodiments, the TPH1 level is determined by ELISA. In some embodiments, the method further comprises administering to the individual an agent that increases brain serotonin level (such as vitamin D, tryptophan, or the combination thereof).

TPH1-generated serotonin increases angiogenesis in endothelial cells. Serotonin stimulates the growth, proliferation and migration of blood vessels. Serotonin produced from TPH1-containing cells can directly promote angiogenesis. Qin et al., Blood 121, 2154-2164 (2013). Further, blocking serotonin receptors in liver can prevent tumor metastasis. Gay et al., Nature Reviews, Cancer 11, 123-134 (2011). TPH1-generated serotonin increases angiogenesis by reducing the expression of matrix metalloproteinase 12 (MMP-12) in tumor-infiltrating macrophages in a mouse model for colon cancer, and serotonin deficiency causes slower growth of tumors by reducing vascularity, thus increasing hypoxia and spontaneous necrosis. Nocito et al., Cancer Research 68, 5152-5158 (2008). We hypothesize that the role of serotonin produced from TPH1 cells has major implications for cancer, which depends on angiogenesis for tumor growth and metastasis, and that vitamin D would prevent tumor metastasis through inhibition of TPH1 and subsequent serotonin-mediated angiogenesis. The present application therefore provides a method for measuring peripheral TPH1 expression and uses thereof as a predictor for risk of relapse and metastasis in breast cancer and other cancers. If TPH1 levels are high then treatment with vitamin D supplementation can be used.

Thus, in some embodiments, there is provided a method of assessing the risk of tumor metastasis (such as breast cancer metastasis) in an individual, comprising determining the peripheral TPH1 level in the individual, wherein a high TPH1 level is indicative that the individual has a high risk of tumor metastasis. In some embodiments, there is provided a method of assessing the risk of tumor metastasis (such as breast cancer metastasis) in an individual, comprising determining the peripheral TPH1 level in the individual using an antibody recognizing TPH1, wherein a high TPH1 level is indicative that the individual has a high risk of developing tumor metastasis. In some embodiments, the TPH1 level is determined by ELISA. In some embodiments, the method further comprises administering to the individual an agent that increases brain serotonin level (such as vitamin D, tryptophan, or the combination thereof).

Antibodies recognizing TPH1 that can be useful for the methods described herein are known in the art, and are described in, for example, Sakowski et al., Brain Res. 2006 Apr. 26; 1085(1):11-8; Bratland et al., Immunobiology 2013, 218(6):899-909; Gershon, Curr. Opin. Endocrinol Diabetes Obes. 2013, 20(1):14-21.

In some embodiments, there is provided a kit comprising: 1) an agent that increases brain serotonin level (such as vitamin D and/or tryptophan, for example, any one of the compositions described herein), and 2) an agent for determining the level of TPH1. In some embodiments, the agent for determining the level of TPH1 is an antibody recognizing TPH1. In some embodiments, the kit further comprises an instruction on use of the agents for any one of the methods described herein.

The level of peripheral TPH1 can also be useful for determining (or aiding assessment in) any one or more of the following: a) probable or likely suitability of an individual to initially receive treatment; b) probable or likely unsuitability of an individual to initially receive treatment(s); c) responsiveness to treatment; d) probable or likely suitability of an individual to continue to receive treatment; e) probable or likely unsuitability of an individual to receive treatment(s); f) adjusting dosage; (g) predicting likelihood of clinical benefits. The present application encompasses any of these methods.

In some embodiments, there is provided a method of supplementing vitamin D to an individual by administering to the individual an effective amount of vitamin D (such as a composition comprising vitamin D and tryptophan), wherein the individual is selected for supplementation based on the peripheral level of TPH1. In some embodiments, there is provided a method of supplementing vitamin D to an individual by administering to the individual an effective amount of vitamin D (such as a composition comprising vitamin D and tryptophan), wherein the peripheral level of TPH1 in the individual is used as a basis for selecting the individual for the supplementation. In some embodiments, the method further comprises determining the level of the peripheral level of TPH1 in the individual prior to the administration of the vitamin D. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

As used herein, “based upon,” “based on,” or “used as a basis” include assessing, determining, or measuring the individual's characteristics as described herein (and preferably selecting an individual suitable for treatment). When peripheral TPH1 level is used as a basis for selection, assessing (or aiding in assessing), measuring, or determining method of treatment as described herein, the TPH1 level is measured before and/or during treatment, and the values obtained can be used, among others, in assessing any one or more of the following: (a) probably or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

In some embodiments, there is provided a method of supplementing vitamin D to an individual by administering to the individual an effective amount of vitamin D (such as a composition comprising vitamin D and tryptophan), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual for supplementation with an effective amount of vitamin D (such as a composition comprising vitamin D and tryptophan), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided supplementing vitamin D to an individual by administering to the individual an effective amount of vitamin D (such as a composition comprising vitamin D and tryptophan), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of supplementing vitamin D to an individual, comprising: (a) selecting an individual having a high peripheral level of TPH1; and (b) administering to the individual an effective amount of vitamin D (such as a composition comprising vitamin D and tryptophan). In some embodiments, there is provided a method of supplementing vitamin D to an individual, comprising: (a) determining the peripheral level of TPH1 in the individual; (b) selecting an individual having a high peripheral level of TPH1; and (c) administering to the individual an effective amount of vitamin D (such as a composition comprising vitamin D and tryptophan).

In some embodiments, a high TPH1 level compared to a reference indicates that a) the individual is more likely to benefit from the vitamin D supplementation or b) the individual is selected for the supplementation. Conversely, a low TPH1 level compared to a reference indicates that a) the individual is less likely to benefit from the vitamin D supplementation or b) the individual is not selected for the supplementation. Thus, in some embodiments, there is provided a method of assessing whether an individual is more likely to benefit or less likely to benefit from supplementation of an effective amount of vitamin D (such as a composition comprising vitamin D and tryptophan), said method comprising assessing the peripheral level of TPH1 in the individual, wherein a high TPH1 level indicates that the individual is more likely to benefit from the supplementation, and wherein a low TPH1 level indicates that the individual is less likely to benefit from the supplementation. In some embodiments, the method further comprises administering to the individual an effective amount of vitamin D and/or tryptophan (such as a composition comprising vitamin D and tryptophan). In some embodiments, the amount of vitamin D is determined based on the peripheral level of TPH1. In some embodiments, the amount of tryptophan is determined based on the peripheral level of TPH1.

In some embodiments, there is provided a method of increasing brain serotonin level (or increasing the brain/peripheral ratio of serotonin) in an individual by administering to the individual an effective amount of an agent that increases brain serotonin level (such as vitamin D and/or tryptophan, for example, any one of the compositions described herein), wherein the individual is selected for treatment based on the peripheral level of TPH1. In some embodiments, there is provided a method of increasing brain serotonin level (or increasing the brain/peripheral ratio of serotonin) in an individual by administering to the individual an effective amount of an agent that increases brain serotonin level (such as vitamin D and/or tryptophan, for example, any one of the compositions described herein), wherein the peripheral level of TPH1 in the individual is used as a basis for selecting the individual for the supplementation. In some embodiments, the method further comprises determining the level of the peripheral level of TPH1 in the individual prior to the administration of the agent. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of increasing brain serotonin level (or increasing the brain/peripheral ratio of serotonin) in an individual by administering to the individual an effective amount of an agent that increases brain serotonin level (such as vitamin D and/or tryptophan, for example, any one of the compositions described herein), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual for treating with an agent that increases brain serotonin level (such as vitamin D and/or tryptophan, for example, any one of the compositions described herein), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of increasing brain serotonin level (or increasing the brain/peripheral ratio of serotonin) in an individual by administering to the individual an effective amount of an agent that increases brain serotonin level (such as vitamin D and/or tryptophan, for example, any one of the compositions described herein), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of increasing brain serotonin level (or increasing the brain/peripheral ratio of serotonin) in an individual, comprising: (a) selecting an individual having a high peripheral level of TPH1; and (b) administering to the individual an effective amount of an agent that increases brain serotonin level (such as vitamin D and/or tryptophan, for example, any one of the compositions described herein). In some embodiments, there is provided a method of increasing brain serotonin level (or increasing the brain/peripheral ratio of serotonin) in an individual, comprising: (a) determining the peripheral level of TPH1 in the individual; (b) selecting an individual having a high peripheral level of TPH1; and (c) administering to the individual an effective amount of an agent that increases brain serotonin level (such as vitamin D and/or tryptophan, for example, any one of the compositions described herein).

In some embodiments, a high TPH1 level compared to a reference indicates that a) the individual is more likely to respond to treatment or b) the individual is selected for treatment. Conversely, a low TPH1 level compared to a reference indicates that a) the individual is less likely to respond to treatment or b) the individual is not selected for treatment. Thus, in some embodiments, there is provided a method of assessing whether an individual is more likely to respond or less likely to respond to treatment, wherein the treatment comprises administering to the individual an effective amount of an agent that increases brain serotonin level (such as vitamin D and/or tryptophan, for example, any one of the compositions described herein), said method comprising assessing the peripheral level of TPH1 in the individual, wherein a high TPH1 level indicates that the individual is more likely to respond to the treatment, and wherein a low TPH1 level indicates that the individual is less likely to respond to the treatment. In some embodiments, the method further comprises administering to the individual an effective amount of an agent that increases brain serotonin level (such as vitamin D and/or tryptophan, for example, any one of the compositions described herein). In some embodiments, the amount of the agent is determined based on the peripheral level of TPH1.

In some embodiments, there is provided a method of treating a brain dysfunction disorder in an individual by administering to the individual an effective amount of an agent that increases brain serotonin level (such as vitamin D and/or tryptophan, for example, any one of the compositions described herein), wherein the individual is selected for treatment based on the peripheral level of TPH1. In some embodiments, there is provided a method of treating a brain dysfunction disorder in an individual by administering to the individual an effective amount of an agent that increases brain serotonin level (such as vitamin D and/or tryptophan, for example, any one of the compositions described herein), wherein the peripheral level of TPH1 in the individual is used as a basis for selecting the individual for the supplementation. In some embodiments, the method further comprises determining the level of the peripheral level of TPH1 in the individual prior to the administration of the agent. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating a brain dysfunction disorder in an individual by administering to the individual an effective amount of an agent that increases brain serotonin level (such as vitamin D and/or tryptophan, for example, any one of the compositions described herein), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual having a brain dysfunction disorder for treating with an agent that increases brain serotonin level (such as vitamin D and/or tryptophan, for example, any one of the compositions described herein), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating a brain dysfunction disorder in an individual by administering to the individual an effective amount of an agent that increases brain serotonin level (such as vitamin D and/or tryptophan, for example, any one of the compositions described herein), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating a brain dysfunction disorder in an individual comprising: (a) selecting an individual having a high peripheral level of TPH1; and (b) administering to the individual an effective amount of an agent that increases brain serotonin level (such as vitamin D and/or tryptophan, for example, any one of the compositions described herein). In some embodiments, there is provided a method of treating a brain dysfunction disorder in an individual comprising: (a) determining the peripheral level of TPH1 in the individual; (b) selecting an individual having a high peripheral level of TPH1; and (c) administering to the individual an effective amount of an agent that increases brain serotonin level (such as vitamin D and/or tryptophan, for example, any one of the compositions described herein).

In some embodiments, there is provided a method of improving pro-social behavior and/or cognitive function in an individual by administering to the individual an effective amount of an agent that increases brain serotonin level (such as vitamin D and/or tryptophan, for example, any one of the compositions described herein), wherein the individual is selected for treatment based on the peripheral level of TPH1. In some embodiments, there is provided a method of improving pro-social behavior and/or cognitive function in an individual by administering to the individual an effective amount of an agent that increases brain serotonin level (such as vitamin D and/or tryptophan, for example, any one of the compositions described herein), wherein the peripheral level of TPH1 in the individual is used as a basis for selecting the individual for the supplementation. In some embodiments, the method further comprises determining the level of the peripheral level of TPH1 in the individual prior to the administration of the agent. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of improving pro-social behavior and/or cognitive function in an individual by administering to the individual an effective amount of an agent that increases brain serotonin level (such as vitamin D and/or tryptophan, for example, any one of the compositions described herein), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual for improving pro-social behavior and/or cognitive function with an agent that increases brain serotonin level (such as vitamin D and/or tryptophan, for example, any one of the compositions described herein), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of improving pro-social behavior and/or cognitive function in an individual by administering to the individual an effective amount of an agent that increases brain serotonin level (such as vitamin D and/or tryptophan, for example, any one of the compositions described herein), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of improving pro-social behavior and/or cognitive function in an individual comprising: (a) selecting an individual having a high peripheral level of TPH1; and (b) administering to the individual an effective amount of an agent that increases brain serotonin level (such as vitamin D and/or tryptophan, for example, any one of the compositions described herein). In some embodiments, there is provided a method of improving pro-social behavior and/or cognitive function in an individual comprising: (a) determining the peripheral level of TPH1 in the individual; (b) selecting an individual having a high peripheral level of TPH1; and (c) administering to the individual an effective amount of an agent that increases brain serotonin level (such as vitamin D and/or tryptophan, for example, any one of the compositions described herein).

In some embodiments, there is provided a method of treating (or preventing) bone abnormality (such as bone loss, osteoporosis, and/or osteomalcia) in an individual by administering to the individual an effective amount of an agent that increases brain serotonin level (such as vitamin D and/or tryptophan, for example, any one of the compositions described herein), wherein the individual is selected for treatment based on the peripheral level of TPH1. In some embodiments, there is provided a method of treating (or preventing) bone abnormality (such as bone loss, osteoporosis, and/or osteomalcia) in an individual by administering to the individual an effective amount of an agent that increases brain serotonin level (such as vitamin D and/or tryptophan, for example, any one of the compositions described herein), wherein the peripheral level of TPH1 in the individual is used as a basis for selecting the individual for the supplementation. In some embodiments, the method further comprises determining the level of the peripheral level of TPH1 in the individual prior to the administration of the agent. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating (or preventing) bone abnormality (such as bone loss, osteoporosis, and/or osteomalcia) in an individual by administering to the individual an effective amount of an agent that increases brain serotonin level (such as vitamin D and/or tryptophan, for example, any one of the compositions described herein), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual for treating (or preventing) hone abnormality (such as bone loss, osteoporosis, and/or osteomalcia) with an agent that increases brain serotonin level (such as vitamin D and/or tryptophan, for example, any one of the compositions described herein), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating (or preventing) bone abnormality (such as bone loss, osteoporosis, and/or osteomalcia) in an individual by administering to the individual an effective amount of an agent that increases brain serotonin level (such as vitamin D and/or tryptophan, for example, any one of the compositions described herein), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of treating (or preventing) bone abnormality (such as bone loss, osteoporosis, and/or osteomalcia) in an individual comprising: (a) selecting an individual having a high peripheral level of TPH1; and (b) administering to the individual an effective amount of an agent that increases brain serotonin level (such as vitamin D and/or tryptophan, for example, any one of the compositions described herein). In some embodiments, there is provided a method of treating (or preventing) bone abnormality (such as bone loss, osteoporosis, and/or osteomalcia) in an individual comprising: (a) determining the peripheral level of TPH1 in the individual; (b) selecting an individual having a high peripheral level of TPH1; and (c) administering to the individual an effective amount of an agent that increases brain serotonin level (such as vitamin D and/or tryptophan, for example, any one of the compositions described herein).

In some embodiments, there is provided a method of reducing risk of tumor metastasis in an individual having cancer by administering to the individual an effective amount of an agent that increases brain serotonin level (such as vitamin D and/or tryptophan, for example, any one of the compositions described herein), wherein the individual is selected for treatment based on the peripheral level of TPH1. In some embodiments, there is provided a method of reducing risk of tumor metastasis in an individual having cancer by administering to the individual an effective amount of an agent that increases brain serotonin level (such as vitamin D and/or tryptophan, for example, any one of the compositions described herein), wherein the peripheral level of TPH1 in the individual is used as a basis for selecting the individual for the supplementation. In some embodiments, the method further comprises determining the level of the peripheral level of TPH1 in the individual prior to the administration of the agent. In some embodiments, the method further comprises comparing the level of the TPH1 with a control. In some embodiments, the individual having a high peripheral level of TPH1 is selected for treatment. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of reducing risk of tumor metastasis in an individual having cancer by administering to the individual an effective amount of an agent that increases brain serotonin level (such as vitamin D and/or tryptophan, for example, any one of the compositions described herein), wherein the individual has a high peripheral level of TPH1. In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of selecting (including identifying) an individual for reducing risk of tumor metastasis with an agent that increases brain serotonin level (such as vitamin D and/or tryptophan, for example, any one of the compositions described herein), wherein the method comprises determining the peripheral level of TPH1 in the individual. In some embodiments, the individual is selected if the individual has a high peripheral level of TPH1 (for example a high level as compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of reducing risk of tumor metastasis in an individual having cancer by administering to the individual an effective amount of an agent that increases brain serotonin level (such as vitamin D and/or tryptophan, for example, any one of the compositions described herein), wherein the individual is selected for treatment based on a high peripheral TPH1 level (for example a high level compared to a control sample). In some embodiments, the peripheral level of TPH1 is determined based on protein expression level. In some embodiments, the peripheral level of TPH1 is determined based on mRNA level. In some embodiments, the peripheral level of TPH1 is determined by an ELISA or immunohistochemistry assay. In some embodiments, the peripheral level of TPH1 (e.g., high or low) by comparing to a control (such as any of the controls described herein). In some embodiments, the peripheral level of TPH1 (e.g., high or low) is determined based on a scoring system.

In some embodiments, there is provided a method of reducing risk of tumor metastasis in an individual having cancer comprising: (a) selecting an individual having a high peripheral level of TPH1; and (b) administering to the individual an effective amount of an agent that increases brain serotonin level (such as vitamin D and/or tryptophan, for example, any one of the compositions described herein). In some embodiments, there is provided a method of reducing risk of tumor metastasis in an individual having cancer comprising: (a) determining the peripheral level of TPH1 in the individual; (b) selecting an individual having a high peripheral level of TPH1; and (c) administering to the individual an effective amount of an agent that increases brain serotonin level (such as vitamin D and/or tryptophan, for example, any one of the compositions described herein).

In some embodiments, a high TPH1 level compared to a reference indicates that a) the individual is more likely to respond to treatment or b) the individual is selected for treatment. Conversely, a low TPH1 level compared to a reference indicates that a) the individual is less likely to respond to treatment or b) the individual is not selected for treatment. Thus, in some embodiments, there is provided a method of assessing whether an individual with a brain dysfunction disorder is more likely to respond or less likely to respond to treatment, wherein the treatment comprises administering to the individual an effective amount of an agent that increases brain serotonin level (such as vitamin D and/or tryptophan, for example, any one of the compositions described herein), said method comprising assessing the peripheral level of TPH1 in the individual, wherein a high TPH1 level indicates that the individual is more likely to respond to the treatment, and wherein a low TPH1 level indicates that the individual is less likely to respond to the treatment. In some embodiments, the method further comprises administering to the individual an effective amount of an agent that increases brain serotonin level (such as vitamin D and/or tryptophan, for example, any one of the compositions described herein). In some embodiments, the amount of vitamin D is determined based on the peripheral level of TPH1. In some embodiments, the amount of tryptophan is determined based on the peripheral level of TPH1.

In some embodiments, a high TPH1 level compared to a reference indicates that the individual is more likely to develop GI inflammation when administered with an agent that increases serotonin level in the peripheral system. Conversely, a low TPH1 level compared to a reference indicates that a) the individual is less likely to respond to develop GI inflammation when administered with an agent that increases serotonin level in the peripheral system. Thus, in some embodiments, there is provided a method of assessing whether an individual is more likely to develop GI inflammation when administered with an agent that increases serotonin level in the peripheral system, said method comprising assessing the peripheral level of TPH1 in the individual, wherein a high TPH1 level indicates that the individual is more likely to develop GI inflammation when administered with an agent that increases serotonin level in the peripheral system, and wherein a low TPH1 level indicates that the individual is less likely to develop GI inflammation when administered with an agent that increases serotonin level in the peripheral system. In some embodiments, the method further comprises administering to the individual an effective amount of an agent that increases serotonin level in the peripheral system.

The peripheral level of TPH1 in an individual can be determined, for example, by analyzing a peripheral sample from an individual. Suitable peripheral samples include, but are not limited to, whole blood, plasma, and peripheral blood lymphocytes. In some embodiments, the peripheral sample is a tissue sample. In some embodiments, the peripheral sample is a cell sample, for example obtained by fine needle aspiration or biopsy. The cells can be pelleted, fixed, and embedded in paraffin prior to analysis. In some embodiments, the cells are flash frozen prior to the analysis.

The methods described herein in some embodiments comprise determining the peripheral level of TPH1 in an individual. In some embodiments, the level is the TPH1 activity level in the peripheral sample. In some embodiments, the level is the TPH1 protein expression level in the peripheral sample. In some embodiments, the level is the TPH1 mRNA level in the peripheral sample. In some embodiments, the level is based on a mutation or polymorphism in the TPH1 gene that correlates with the protein or mRNA level of TPH1.

The peripheral TPH1 level may be a high or a low level as compared to a control sample. In some embodiments, the peripheral TPH1 level in an individual is compared to the TPH1 level in a control sample. In some embodiments, the peripheral TPH1 level in an individual is compared to the TPH1 level in multiple control samples. In some embodiments, multiple control samples are used to generate a statistic that is used to classify the peripheral level of TPH1 as high or low.

The classification or ranking of the TPH1 peripheral level (e.g., high or low) may be determined relative to a statistical distribution of control levels. In some embodiments, the classification or ranking is relative to a control sample (e.g., a non-peripheral sample, or a peripheral sample previously obtained) obtained from the individual. In some embodiments, the peripheral level of TPH1 is classified or ranked relative to a statistical distribution of control levels.

Control samples can be obtained using the same sources and methods as non-control samples. In some embodiments, the control sample is obtained from a different individual (for example an individual not having a brain dysfunction disorder and/or an individual sharing similar ethnic, age, and gender identity). In some embodiments, the control is a non-peripheral sample from the same individual. In some embodiments, the control is a peripheral sample from the same individual that was previously obtained. In some embodiments, multiple control samples (for example from different individuals) are used to determine a range of TPH1 levels in a peripheral sample. In some embodiments, the control sample is a cultured tissue or cell that has been determined to be a proper control. In some embodiments, a clinically accepted normal level in a standardized test is used as a control level for determining peripheral level of TPH1. In some embodiments, the peripheral level of TPH1 is classified as high, medium, or low according to a scoring system, such as an ELISA or immunohistochemistry-based scoring system.

In some embodiments, the peripheral TPH1 level is determined by measuring the peripheral level of TPH1 in an individual and comparing to a control or reference (e.g., the median level for a given population or level of a second individual). For example, if the peripheral level of TPH1 in the individual is above the median level of the chosen population, that individual is determined to have high peripheral expression of TPH. Alternatively, if the peripheral level of TPH1 in the individual is below the median level of the chosen population, that individual is determined to have low peripheral expression of TPH1.

In some embodiments, the peripheral TPH1 level is determined by obtaining a statistical distribution of TPH1 levels.

In some embodiments, bioinformatics methods are used for the determination and classification of peripheral TPH1 level. Numerous bioinformatics approaches have been developed to assess gene set expression profiles using gene expression profile data. Methods include but are not limited to Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007). In some embodiments, the TPH1 level is determined along with at least 1, 2, 3, 4, 5, 10, 20, 30, 40, 50, 60, 70, 80, 90, 100, 200, 300, 400, or more other biomarkers.

In some embodiments, mRNA level is determined, and a low level is an mRNA level less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the level obtained from a control. In some embodiments, high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

In some embodiments, protein expression level is determined, for example by ELISA or immunohistochemistry. For example, the criteria for low or high levels can be made based on the number of positive staining cells and/or the intensity of the staining, for example by using an antibody that specifically recognizes the nucleoside transporter protein. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense appositive control staining. In some embodiments, TPH1 protein level is determined, and a low level is a protein level less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the level obtained from a control. In some embodiments, high level is a protein level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

Further provided herein are methods of directing treatment of a brain dysfunction disorder by delivering a sample to a diagnostic lab for determination of peripheral level of TPH1; providing a control sample with a known level of a peripheral TPH1; providing an antibody recognizing TPH1; subjecting the sample and control sample to binding by the antibody, and/or detecting a relative amount of antibody binding, wherein the TPH1 level of the sample is used to provide a conclusion that a patient should receive a treatment with any one of the methods described herein.

Also provided are methods of directing treatment of a disease, further comprising reviewing or analyzing data relating to the level of TPH1 in a peripheral sample; and providing a conclusion to an individual about the likelihood or suitability of the individual to respond to a treatment, a healthcare provider or a healthcare manager, the conclusion being based on the review or analysis of data. In one aspect of the invention a conclusion is the transmission of the data over a network.

### Methods Based on TPH1 Level in a Pregnant Individual

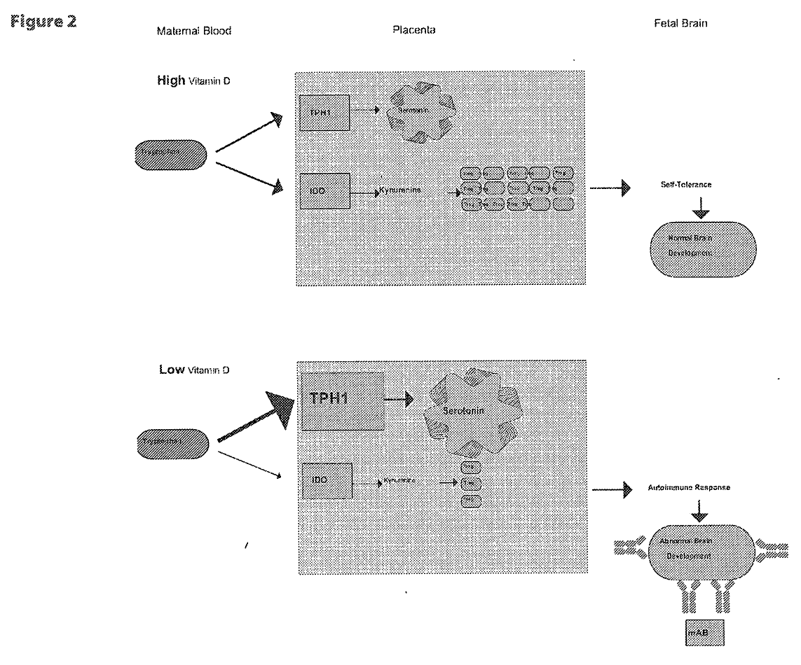

Another aspect of the present invention is based on the finding that dysregulation of tryptophan metabolism, and thus kynurenines, could result from vitamin D insufficiency due to overexpression of TPH1 in the placenta. This could cause an imbalance in tryptophan catabolism in the placenta, resulting in too much serotonin and too little kynurenine, thus leading to an autoimmune response attacking the fetus and fetal brain, increasing the risk of the newborn having autistic spectrum disorder and other brain dysfunction disorders. FIG. 2 provides a model of the maternal contribution to autoimmune antibodies in the fetal brain. (A) Vitamin D sufficiency during pregnancy allows normal tryptophan metabolism in the placenta, producing serotonin and kynurenines. Kynurenines generate regulatory T cells (Tregs), which allow self-tolerance and normal fetal brain development. (B) Under vitamin D insufficiency, tryptophan hydroxylase 1 (TPH1) is overexpressed. This shunts tryptophan away from indoleamine 2,3-dioxygenase (IDO), blunting the production of Tregs and causing maternal autoantibodies to attack the fetal brain, which leads to abnormal brain development.

The present application thus in some embodiments provides placenta TPH1 as an accurate and specific indicator of the tryptophan catabolism in the placenta. By determining the level of TPH1 in the placenta, one can detect the imbalance between serotonin and kynurenine in the placenta and consequences thereof. As a high peripheral level of TPH1 in a pregnant individual is indicative of a high placenta level of TPH1, one can also detect the imbalance between serotonin and kynurenine in the placenta and consequences thereof by determining the peripheral TPH1 level in the pregnant individual.

Thus, in some embodiments, there is provided a method of assessing the serotonin/kynurenine balance in the placenta of an individual, comprising determining the placenta (or peripheral) TPH1 level in the individual, wherein a high TPH1 level is indicative that the individual has imbalanced serotonin/kynurenine levels in the placenta. In some embodiments, there is provided a method of assessing the serotonin/kynurenine balance in the placenta of an individual, comprising determining the placenta (or peripheral) TPH1 level in the individual using an antibody recognizing TPH1, wherein a high TPH1 level is indicative that the individual has an imbalanced serotonin/kynurenine levels in the placenta.

The level of placenta (or peripheral) TPH1 in a pregnant individual can also be useful for determining (or aiding assessment in) any one or more of the following: a) risk of the pregnant individual developing autoimmune reaction against the fetus; (2) risk of the pregnant individual having fetal loss; (3) risk of the pregnant individual developing an autoimmune disease such as lupus; and (4) whether the pregnant individual is in need of vitamin D supplementation.

The level of placenta (or peripheral) TPH1 in a pregnant individual can also be useful for determining (or aiding assessment in) risks in the child developed from the fetus. The risks include, but are not limited to: (I) risk of the child developing one or more brain dysfunction disorder (including for example autistic spectrum disorder, schizophrenia, and any other disorders described herein); (2) risk of the child developing an autoimmune disorder of the central nervous system (such as multiple sclerosis).

In some embodiments, the pregnant individual is at least about 1, 2, 3, 4, 5, 6, 7, 8, 9, or 10 months pregnant. In some embodiments, the pregnant individual is at least about 30, 35, 40, 45, or 50 years old.

In some embodiments, there is provided a method of decreasing placenta serotonin level in a pregnant individual (and optionally increasing the level of kynurenine in the placenta), comprising administering (such as orally administering) to the individual an effective amount of vitamin D (such as a composition comprising vitamin D and tryptophan), wherein the placenta (or peripheral) level of TPH1 in the individual is used as a basis for selecting the individual for the administration. In some embodiments, there is provided a method of modulating the serotonin/kynurenine levels in a pregnant individual (and optionally increasing the level of kynurenine in the placenta), comprising administering (such as orally administering) to the individual an effective amount of vitamin D (such as a composition comprising vitamin D and tryptophan), wherein the placenta (or peripheral) level of TPH1 in the individual is used as a basis for selecting the individual for the administration. In some embodiments, the method further comprises determining the level of the placenta (or peripheral) level of TPH1 in the individual prior to the administration of the vitamin D. In some embodiments, the method further comprises comparing the level of the TPH1 with a control. In some embodiments, the level of the TPH1 is classified as high, medium, and low according to a scoring system.

In some embodiments, there is provided a method of selecting (including identifying) a pregnant individual for treating with vitamin D (such as a composition comprising vitamin D and tryptophan), wherein the method comprises determining the placenta (or peripheral) level of TPH1 in the individual. In some embodiments, the individual is selected if the individual has a high placenta (or peripheral) level of TPH1 (for example a high level as compared to a control sample). In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops a brain dysfunction disorder (such as autism or schizophrenia), comprising administering to the individual an effective amount of vitamin D (such as a composition comprising vitamin D and tryptophan), wherein the individual is selected for treatment based on the placenta (or peripheral) level of TPH1. In some embodiments, the individual having a high placenta (or peripheral) level of TPH1 is selected for treatment. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops a brain dysfunction disorder (such as autism or schizophrenia), comprising administering to the individual an effective amount of vitamin D (such as a composition comprising vitamin D and tryptophan), wherein the individual has a high placenta (or peripheral) level of TPH1. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops a brain dysfunction disorder (such as autism or schizophrenia), comprising administering to the individual an effective amount of vitamin D (such as a composition comprising vitamin D and tryptophan), wherein the individual is selected for treatment based on a high placenta (or peripheral) TPH1 level (for example a high level compared to a control sample). In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops a brain dysfunction disorder (such as autism or schizophrenia), comprising: (a) selecting a pregnant individual having a high placenta (or peripheral) level of TPH1; and (b) administering to the individual an effective amount of vitamin D (such as a composition comprising vitamin D and tryptophan). In some embodiments, there is provided a method of preventing a pregnant individual from having a child who develops a brain dysfunction disorder (such as autism or schizophrenia), comprising: (a) determining the placenta (or peripheral) level of TPH1 in the individual; (b) selecting an individual having a high placenta (or peripheral) level of TPH1; and (c) administering to the selected individual an effective amount of vitamin D (such as a composition comprising vitamin D and tryptophan).

In some embodiments, there is provided a method of preventing a pregnant individual from having a child who develops an autoimmune disorder of the central nervous system (such as multiple sclerosis), comprising administering to the individual an effective amount of vitamin D (such as a composition comprising vitamin D and tryptophan), wherein the individual is selected for treatment based on the placenta (or peripheral) level of TPH1. In some embodiments, the individual having a high placenta (or peripheral) level of TPH1 is selected for treatment. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops an autoimmune disorder of the central nervous system (such as multiple sclerosis), comprising administering to the individual an effective amount of vitamin D (such as a composition comprising vitamin D and tryptophan), wherein the individual has a high placenta (or peripheral) level of TPH1. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops an autoimmune disorder of the central nervous system (such as multiple sclerosis), comprising administering to the individual an effective amount of vitamin D (such as a composition comprising vitamin D and tryptophan), wherein the individual is selected for treatment based on a high placenta (or peripheral) TPH1 level (for example a high level compared to a control sample). In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having a child who develops an autoimmune disorder of the central nervous system (such as multiple sclerosis), comprising: (a) selecting a pregnant individual having a high placenta (or peripheral) level of TPH1; and (b) administering to the individual an effective amount of vitamin D (such as a composition comprising vitamin D and tryptophan). In some embodiments, there is provided a method of preventing a pregnant individual from having a child who develops an autoimmune disorder of the central nervous system (such as multiple sclerosis), comprising: (a) determining the placenta (or peripheral) level of TPH1 in the individual; (b) selecting an individual having a high placenta (or peripheral) level of TPH1; and (c) administering to the selected individual an effective amount of vitamin D (such as a composition comprising vitamin D and tryptophan).

In some embodiments, there is provided a method of preventing a pregnant individual from having fetal loss, comprising administering to the individual an effective amount of vitamin D (such as a composition comprising vitamin D and tryptophan), wherein the individual is selected for treatment based on the placenta (or peripheral) level of TPH1. In some embodiments, the individual having a high placenta (or peripheral) level of TPH1 is selected for treatment. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having fetal loss, comprising administering to the individual an effective amount of vitamin D (such as a composition comprising vitamin D and tryptophan), wherein the individual has a high placenta (or peripheral) level of TPH1. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having fetal loss, comprising administering to the individual an effective amount of vitamin D (such as a composition comprising vitamin D and tryptophan), wherein the individual is selected for treatment based on a high placenta (or peripheral) TPH1 level (for example a high level compared to a control sample). In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from having fetal loss, comprising: (a) selecting a pregnant individual having a high placenta (or peripheral) level of TPH1; and (b) administering to the individual an effective amount of vitamin D (such as a composition comprising vitamin D and tryptophan). In some embodiments, there is provided a method of preventing a pregnant individual from having fetal loss, comprising: (a) determining the placenta (or peripheral) level of TPH1 in the individual; (b) selecting an individual having a high placenta (or peripheral) level of TPH1; and (c) administering to the selected individual an effective amount of vitamin D (such as a composition comprising vitamin D and tryptophan).

In some embodiments, there is provided a method of preventing a pregnant individual from developing an autoimmune disease (such as lupus), comprising administering to the individual an effective amount of vitamin D (such as a composition comprising vitamin D and tryptophan), wherein the individual is selected for treatment based on the placenta (or peripheral) level of TPH1. In some embodiments, the individual having a high placenta (or peripheral) level of TPH1 is selected for treatment. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from developing an autoimmune disease (such as lupus), comprising administering to the individual an effective amount of vitamin D (such as a composition comprising vitamin D and tryptophan), wherein the individual has a high placenta (or peripheral) level of TPH1. In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from developing an autoimmune disease (such as lupus), comprising administering to the individual an effective amount of vitamin D (such as a composition comprising vitamin D and tryptophan), wherein the individual is selected for treatment based on a high placenta (or peripheral) TPH1 level (for example a high level compared to a control sample). In some embodiments, the placenta (or peripheral) level of TPH1 is determined based on protein expression level. In some embodiments, the placenta (or peripheral) level of TPH1 is determined based on mRNA level. In some embodiments, the placenta (or peripheral) level of TPH1 is determined by an ELISA or immunohistochemistry assay. In some embodiments, the placenta (or peripheral) level of TPH1 (e.g., high or low) by comparing to a control (such as any of the controls described herein). In some embodiments, the placenta (or peripheral) level of TPH1 (e.g., high or low) is determined based on a scoring system.

In some embodiments, there is provided a method of preventing a pregnant individual from developing an autoimmune disease (such as lupus), comprising: (a) selecting a pregnant individual having a high placenta (or peripheral) level of TPH1; and (b) administering to the individual an effective amount of vitamin D (such as a composition comprising vitamin D and tryptophan). In some embodiments, there is provided a method of preventing a pregnant individual from developing an autoimmune disease (such as lupus), comprising: (a) determining the placenta (or peripheral) level of TPH1 in the individual; (b) selecting an individual having a high placenta (or peripheral) level of TPH1; and (c) administering to the selected individual an effective amount of vitamin D (such as a composition comprising vitamin D and tryptophan).

In some embodiments, a high TPH1 level compared to a reference indicates that a) the individual is more likely to respond to treatment or b) the individual is selected for treatment. Conversely, a low TPH1 level compared to a reference indicates that a) the individual is less likely to respond to treatment or b) the individual is not selected for treatment. Thus, in some embodiments, there is provided a method of assessing whether an individual with autistic spectrum disorder is more likely to respond or less likely to respond to treatment, wherein the treatment comprises vitamin D (such as a composition comprising vitamin D and tryptophan), said method comprising assessing the placenta (or peripheral) level of TPH1 in the individual, wherein a high TPH1 level indicates that the individual is more likely to respond to the treatment, and wherein a low TPH1 level indicates that the individual is less likely to respond to the treatment. In some embodiments, the method further comprises administering to the individual an effective amount of vitamin D and/or tryptophan (such as a composition comprising vitamin D and tryptophan). In some embodiments, the amount of vitamin D is determined based on the placenta (or peripheral) level of TPH1. In some embodiments, the amount of tryptophan is determined based on the placenta (or peripheral) level of TPH1.

The methods described herein in some embodiments comprise determining the placenta (or peripheral) level of TPH1 in an individual. In some embodiments, the level is the TPH1 activity level in the placenta (or peripheral) sample. In some embodiments, the level is the TPH1 protein expression level in the placenta (or peripheral) sample. In some embodiments, the level is the TPH1 mRNA level in the placenta (or peripheral) sample. In some embodiments, the level is based on a mutation or polymorphism in the TPH1 gene that correlates with the protein or mRNA level of TPH1.

The placenta (or peripheral) TPH1 level may be a high or a low level as compared to a control sample. In some embodiments, the placenta (or peripheral) TPH1 level in an individual is compared to the TPH1 level in a control sample. In some embodiments, the placenta (or peripheral) TPH1 level in an individual is compared to the TPH1 level in multiple control samples. In some embodiments, multiple control samples are used to generate a statistic that is used to classify the placenta (or peripheral) level of TPH1 as high or low.

The classification or ranking of the TPH1 placenta (or peripheral) level (e.g., high or low) may be determined relative to a statistical distribution of control levels. In some embodiments, for example, the classification or ranking of the placenta sample is relative to a control sample (e.g., a non-placenta sample, or a placenta sample previously obtained) obtained from the individual. In some embodiments, the placenta (or periphernal) level of TPH1 is classified or ranked relative to a statistical distribution of control levels.

Control samples can be obtained using the same sources and methods as non-control samples. In some embodiments, the control sample is obtained from a different individual (for example an individual sharing similar ethnic or age). In some embodiments, the control is a non-placenta sample from the same individual. In some embodiments, the control is a placenta sample from the same individual that was previously obtained. In some embodiments, multiple control samples (for example from different individuals) are used to determine a range of TPH1 levels in a placenta (or peripheral) sample. In some embodiments, the control sample is a cultured tissue or cell that has been determined to be a proper control. In some embodiments, a clinically accepted normal level in a standardized test is used as a control level for determining placenta (or peripheral) level of TPH1. In some embodiments, the placenta (or peripheral) level of TPH1 is classified as high, medium, or low according to a scoring system, such as an ELISA or immunohistochemistry-based scoring system.

In some embodiments, the placenta (or peripheral) TPH1 level is determined by measuring the placenta (or peripheral) level of TPH1 in an individual and comparing to a control or reference (e.g., the median level for a given population or level of a second individual). For example, if the placenta (or peripheral) level of TPH1 in the individual is above the median level of the chosen population (e.g., a population of pregnant individuals with adequate vitamin D serum levels), that individual is determined to have high placenta (or peripheral) expression of TPH1. Alternatively, if the placenta (or peripheral) level of TPH1 in the individual is below the median level of the chosen population, that individual is determined to have low placenta (or peripheral) expression of TPH1.

In some embodiments, the placenta (or peripheral) TPH1 level is determined by obtaining a statistical distribution of TPH1 levels.

In some embodiments, bioinformatics methods are used for the determination and classification of placenta (or peripheral) TPH1 level. Numerous bioinformatics approaches have been developed to assess gene set expression profiles using gene expression profile data.

In some embodiments, mRNA level is determined, and a low level is an mRNA level less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the level obtained from a control. In some embodiments, high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

In some embodiments, protein expression level is determined, for example by ELISA or immunohistochemistry. For example, the criteria for low or high levels can be made based on the number of positive staining cells and/or the intensity of the staining, for example by using an antibody that specifically recognizes the nucleoside transporter protein. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than positive control staining. In some embodiments, TPH1 protein level is determined, and a low level is a protein level less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the level obtained from a control. In some embodiments, high level is a protein level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

Further provided herein are methods of directing treatment by delivering a sample to a diagnostic lab for determination of placenta (or peripheral) level of TPH1; providing a control sample with a known level of a placenta (or peripheral) TPH1; providing an antibody recognizing TPH1; subjecting the sample and control sample to binding by the antibody, and/or detecting a relative amount of antibody binding, wherein the TPH1 level of the sample is used to provide a conclusion that a patient should receive a treatment with any one of the methods described herein.

Also provided are methods of directing treatment of a disease, further comprising reviewing or analyzing data relating to the level of TPH1 in a placenta (or peripheral) sample; and providing a conclusion to an individual about the likelihood or suitability of the individual to respond to a treatment, a healthcare provider or a healthcare manager, the conclusion being based on the review or analysis of data. In one aspect of the invention a conclusion is the transmission of the data over a network.

Administration of Vitamin D and/or Tryptophan

The various methods described above involve administration of vitamin D and/or tryptophan. In some embodiments, the vitamin D and/or tryptophan (such as the vitamin D/tryptophan compositions described herein) are administered at least 1×, 2×, 3×, 4×, 5×, 6×, or 7× per week. In some embodiments, the vitamin D and/or tryptophan (such as the vitamin D/tryptophan compositions described herein) are administered once a day. In some embodiments, the vitamin D and/or tryptophan (such as the vitamin D/tryptophan compositions described herein) are administered twice a day. In some embodiments, the vitamin D and/or tryptophan (such as the vitamin D/tryptophan composition described herein) are administered thrice daily, 4× daily, or more frequently. In some embodiments, the composition is administered along with food. In some embodiments, the vitamin D and/or tryptophan (such as the vitamin D/tryptophan composition described herein) is administered on an empty stomach. In some embodiments, the vitamin D and/or tryptophan (such as the vitamin D/tryptophan composition described herein) are administered in conjunction with other agents. In some embodiments, the vitamin D and/or tryptophan (such as the vitamin D/tryptophan compositions described herein) are administered during the day. In some embodiments, the vitamin D and/or tryptophan (such as the vitamin D/tryptophan compositions described herein) are administered in conjunction with water.

The amounts of vitamin D, tryptophan, and other components described herein may be adjusted based on body weight of the individual, and can be administered at once, or may be divided into a number of smaller doses to be administered at predetermined intervals of time.

In some embodiments, the dose of vitamin D is at least about 500 IU (such as at least about any of 600, 700, 800, 900, 1000, 1500, 2000, 2500, 3000, 3500, or 4000 IU) per day. In some embodiments, the dose of vitamin D is any of about 500 to about 1000, about 1000 to about 1500, about 1500 to about 2000, about 2000 to about 2500, about 2500 to about 3000, about 3000 to about 3500, about 3500 to about 4000 IU. In some embodiments, the dose of vitamin D is about 30 to about 200 IU/kg/day, including for example about 40 to about 150 IU/kg/day, about 50 to about 100 IU/kg/day. In some embodiments, the dose of vitamin D is at least about 30 IU/kg/day, including for example at least about any of 40, 50, 60, 70, 80, 90, or 100 IU/kg/day. In some embodiments, the dose of vitamin D is at least about 30 IU/kg/day and no more than about any of 50, 60, 70, 80, 90, 100, 110, 120, 130, 140, 150, 160, 170, 180, 190, or 200 IU/kg/day.

In some embodiments, the dose of tryptophan is at least about 100 mg (such as at least about any of 200, 500, 800, 1000, 2000, 3000, 4000, 5000, or 6000 mg) per day. In some embodiments, the dose of tryptophan is at least about 100 to about 500, about 500 to about 1000, about 1000 to about 1500, about 1500 to about 2000, about 2000 to about 2500, about 2500 to about 3000, about 3000 to about 3500, about 3500 to about 4000, about 4000 to about 4500, about 4500 to about 5000, about 5000 to about 5500, or about 5500 to about 6000 mg. In some embodiments, the dose of tryptophan is about 30 to about 200 mg/kg/day, including for example about 40 to about 150 mg/kg/day, about 50 to about 100 mg/kg/day. In some embodiments, the dose of vitamin D is at least about 30 mg/kg/day, including for example at least about any of 40, 50, 60, 70, 80, 90, or 100 mg/kg/day. In some embodiments, the dose of vitamin D is at least about 30 IU/kg/day and no more than about any of 50, 60, 70, 80, 90, 100, 110, 120, 130, 140, 150, 160, 170, 180, 190, or 200 mg/kg/day.

In some embodiments, the dose of LCPUFA is about 30 to about 200 mg/kg/day, including for example about 40 to about 150 mg/kg/day, about 50 to about 100 mg/kg/day. In some embodiments, the dose of vitamin D is at least about 30 mg/kg/day, including for example at least about any of 40, 50, 60, 70, 80, 90, or 100 mg/kg/day. In some embodiments, the dose of vitamin D is at least about 30 IU/kg/day and no more than about any of 50, 60, 70, 80, 90, 100, 110, 120, 130, 140, 150, 160, 170, 180, 190, or 200 mg/kg/day.

In some embodiments, the compositions described herein are administered orally. In some embodiments, the compositions described herein are administered parenterally. In some embodiments the compositions described herein are administered intravenously. In some embodiments, the compositions described herein are administered subcutaneously. In some embodiments, the compositions are administered through nasal spray.

### EXAMPLES

### Example 1. Identification of a Unifying Mechanism Linking Serotonin and Vitamin D Levels

Vitamin D-regulated transcription occurs both by gene activation and repression. Whitfield 2005, Molecular Control of Gene Transcription and Novel Bioactions (Elsevier, Inc.) 2^{nd} Ed. Upon binding of vitamin D to the vitamin D receptor (VDR), the VDR heterodimerizes with the retinoid X receptor (RXR), and triggers the VDR to recognize vitamin D response elements (VDREs) in DNA sequences of vitamin D-regulated genes. Haussler et al., 2011, Best Pract. Res. Clin. Endocrinol Metab 25(4):543-559. It has been previously demonstrated that the VDRE sequence alone can determine whether the VDR-RXR heterodimer activates or represses transcription possibly by inducing a conformational change that favors recruitment of either co-activators or co-repressors; however, the exact mechanism was unclear. Haussler et al. The most common VDREs are composed of two hexanucleotide direct repeats consisting of (Pu)G(G/T)TCA, (where Pu is a purine), separated by a three nucleotide space, called DR3 subtype. The optimal VDRE for transcriptional activation is (Pu)GGTCA for the 5′ half site and (Pu)GTTCA for the 3′ half site. Variations in the sequence of the DR3 subtype of VDRE are common, with one to three base substitutions usually occurring in purines in either half-site. Strom et al. 1992, Proc. Soc. Exp. Biol. Med. 199(3):369-371; Cao et al., 1993, J. Biol. Chem. 268 (36):27371-27380. Multiple distal activating VDREs in the gene can synergize to upregulate gene transcription, which is thought to occur through chromatin looping thereby inducing a conformational change replacing bound co-repressors with co-activators. Haussler et al.; Xu 2005, Biochemistry and Cell Biology=Biochimie et Biologic Cellulaire 83(4):418-28. Transcriptionally-repressing VDREs consist of distinct base substitutions that differ from substitutions that are present in activating VDREs. Haussler et al. 2008, Nutrition Reviews 66 (10 Suppl 2):S98-112. Repressing VDREs consist of substitutions in either the 5′ or 3′ repeat, or both, and typically occur in pyrimidines. VDRE-mediate repression may occur by multiple mechanisms that are less defined than activation. Many genes that are transcriptionally repressed by vitamin D have multiple repressing and activating VDERs. It has been shown that the activating VDERs, but not the repressing VDERs, binds to the VDR-RXR heterodimer and loops around to the repressing VDER to replace co-activators with co-repressors. Kato et al 2007, 16(4):297-304; Turunen et al., 2007, Nucleic Acids Research 35(8):2734-2747.

By examining the specific sequences in the VDREs of both TPH1 and TPH2, we revealed that TPH2 has two distal activating VDRE sequences that are associated with transcriptional activation. In contrast, TPH1 contains a distal repressing VDRE that is only associated with gene repression and a proximal VDRE with variations that have been observed in activating VDREs. Since TPH2 has two activating VDREs that have been associated with transcriptional activation, it is likely to be transcriptionally activated by vitamin D. The repressing VDRE in TPH1 is identical to that of rat parathyroid hormone-related peptide, which is downregulated by vitamin D despite also possessing an activating VDRE in the promoter region. Since TPH1's distal VDRE has a substitution that is known to be exclusively associated with regressing regulation by vitamin D, it likely indicates transcriptional repression. Thus, we believe that the production of serotonin by these two enzyme isoforms would be regulated in opposite directions.

Our hypothesis on the differential regulation of TPH1 and TPH2 by vitamin D on serotonin production provides an elegant and unifying explanation of many different previous observations. First, serotonin in the blood, which is produced from TPH1, is lowest in summer months and highest in winter, whereas brain serotonin, which is generated from TPH2, is highest in summer months and lowest in winter months. These data are in agreement with the seasonal variation in serum vitamin D levels that have been observed. Our hypothesis explains the seasonal variation of serotonin levels in brain as compared to peripheral tissues. Second, there is an inverse relationship between serum vitamin D levels and melatonin, which is made from TPH-1-mediated serotonin in the pineal gland. It has been demonstrated that with increasing doses of vitamin D supplementation there is a dose-dependent decrease in melatonin production. This suggests that the inverse relationship between vitamin D levels and melatonin may be due to vitamin D-mediated transcriptional repression of TPH1. Third, TPH1 mRNA expression is lowest during the day and highest during the night, whereas TPH2 is highest in the day and lowest in the night. Together, all of these observations can be explained by a novel mechanism by which vitamin D transcriptionally represses TPH1 and activates TPH2, thereby inversely affecting serotonin production in peripheral tissues relative to production in the brain.

It has previously been shown that the genes encoding the oxytocin/neurophysin I prepropeptide (OXT) and the oxytocin receptor (OXTR) contain multiple VDREs. Wang, 2005, Mol. Endocrinol 19(11):2685-2695. We confirmed that OXT contains a proximal and three distal VDREs and OXTR has one distal VDRE. After careful examination of the VDRE sequences, we found that these putative VDREs mostly appear to be consistent with transcriptional activation, suggesting that vitamin D could regulate both the production of the oxytocin hormone and the response to it. FIG. 1. OXT contains three putative activating VDREs and one repressing VDRE. The four different VDREs present in OXT likely modulate the production oxytocin in different tissues. OXTR contains a putative VDRE that may be associated with activation. FIG. 1. Consistent with the hypothesis that vitamin D regulates these oxytocin-related genes, it has previously been shown that the vitamin D receptor co-localizes with oxytocin in hypothalamic neurons. We thus hypothesize that vitamin D could modulate oxytocin synthesis as well as of the response to the neuropeptide itself in different tissues, with important implications for benefiting social behavior.

Putative VDREs have previously been identified in the genes encoding two receptors for the vasopressin peptide, AVPR1A and AVPR1B. Wang T T, et al. (2005), Mol Endocrinol 19(11):2685-2695. After careful examination of the VDRE sequences, we found that these VDREs are consistent with transcriptional activation, indicating that vitamin D hormone may also modulate the expression of these critical vasopressin receptors. FIG. 1. We thus hypothesize that vitamin D could modulate vasopressin receptor synthesis in different tissues, with important implications for benefiting social behavior.

FIG. 1 provides representative DR3 vitamin D response element (VDRE) subtypes in TPH1, TPH2, Oxytocin, Oxytocin receptor, arginine vasopressin receptor 1A (AVPR1A) and arginine vasopressin 1B (AVPR1B). Activating and repressing DR3 VDREs in TPH1, TPH2, OXT, OXTR, AVPR1A, and AVPR1B are shown in the figure. The most common DR3 vitamin D response element (VDRE) for activation is represented as a 5′- and 3′-hexamer separated by three nucleotides (spacer). Known substitutions in either the 5′- or 3′-half sites associated with transcriptional activation or repression are denoted in bold italic. Substitutions in purines are commonly associated with activation and substitutions in pyrimadines are repressing. The activating or repressing VDREs for tryptophan hydroxylase 1 (TPH1), tryptophan hydroxylase 2 (TPH2), oxytocin-neurophysin I prepropeptide (OXT), oxytocin receptor (OXTR), arginine vasopressin receptor 1A (AVPR1A) and arginine vasopressin receptor 1B (AVPR1B) are shown with base substitutions in bold italic. *This substitution occurs in an existing purine and is most likely associated with activation.

### Example 2. Identification of Mechanism Underlying Maternal Autoimmunity

Dysregulation of tryptophan metabolism during pregnancy, which could result from vitamin D insufficiency, could also lead to an altered balance of maternal immunity. Tryptophan plays an important role in regulating the autoimmune response during pregnancy through its conversion to kynurenine in the placenta. Munn, 1998, Science 281 (5380):1191-1193; Mellor et al. 2001, Nature Immunology, 2(1):64-68. There are essentially three competing fates for tryptophan: use in protein synthesis, metabolic conversion by TPH to serotonin, and metabolic conversion by the enzyme indoleamine 2,3-dioxygenase (IDO) to kynurenine. Kynurenine in the placenta is required during pregnancy to prevent a general autoimmune response by generating regulatory T cells (Tregs), which maintain tolerance to self-antigens and keep autoimmunity under control by mediating maternal tolerance to the fetal-derived placenta. See FIG. 2.

The placenta expresses both TPH1 and IDO. Munn et al.; Bonnin et al. Low maternal vitamin D levels may result in increased placental expression of TPH1 due to its being regulated by a vitamin D-repressible VDRE. This hypothesis explains the fact that mothers of autistic children also display abnormally high serotonin levels in peripheral white blood cells that express TPH1. Anderson 2002, Journal of the American Academy of Child and Adolescent Psychiatry 41(12):1513-1516; Cook et al. 1990, The Journal of Neuropsychiatry and Clinical Neurosciences 2(3):268-274; Leboyer et al. 2999, Biological Psychiatry 45(2):158-163; Hranilovic 2007, Journal of Autism and Developmental Disorders 37(1): 1934-1940. Since TPH1 has a three-fold tighter tryptophan-binding affinity than IDO, such increased TPH1 expression would result in aberrant tryptophan catabolism. In addition, TPH1 has a fairly long half-life of approximately 1 hour, whereas IDO protein levels are normally very low and thus would have relatively lower activity. Pallotta M T, et al. (2011) Nature immunology 12(9):870-878; Nakamura K, et al. (2006), the Journal of Neuroscience: the official journal of the Society for Neuroscience 26(2):530-534; Batabyal D & Yeh S R (2007), Journal of the American Chemical Society 129(50):15690-15701. We hypothesize that elevated expression of TPH1 as a consequence of vitamin D insufficiency causes TPH1 activity to act as a tryptophan trap, thus shunting tryptophan away from the kynurenine pathway and decreasing placental production of kynurenines and Tregs. Vitamin D supplementation has been shown to significantly increase Tregs in peripheral blood. Chambers E S & Hawrylowicz C M (2011), 11(1):29-36. We propose a mechanism for the vitamin D-dependent increase in Tregs is due to its suppression of TPH1, thus allowing tryptophan to be catabolized to kyurenine. In summary, low maternal vitamin D during pregnancy would cause an imbalance in tryptophan catabolism in the placenta resulting in too much serotonin and too little kynurenine, thus leading to an autoimmune response attacking the fetus and fetal brain, tipping the balance towards inflammation and autoimmunity.

### Example 3. Compositions Comprising Vitamin D and Tryptophan

Pills each containing 4,000 IU of vitamin D, 1.5 grams of tryptophan, 3 grams of omega-3 are made. The pills also optionally contain vitamin B6 and BH4. The pills are administered to an individual weighing about 150 lbs on a daily basis during the day time with one pill per day.

### Example 4. Methods of Treating Autistic Spectrum Disorder

Children of about 7 years old with autistic spectrum disorder that do not engage in social interaction are given the vitamin D/tryptophan/omega-3 formulation of Example 3 at the dose of one pill per day and it improves their social interaction with others so they spend more time engaging in social behavior and feel less social anxiety. It improves their ability to interpret facial expressions of sadness, anger, and fear so they know what another person is feeling and can react accordingly. It improves repetitive hand flapping, arm waving, and rocking behaviors and broadens the types of foods they eat. It decreases tantrums and aggressive outbursts so they are more agreeable.

### Example 5. Determination of TPH1 in Peripheral Tissue

TPH1 expression levels are determined from peripheral blood cells by ELISA analysis from a pregnant mother. The results show that TPH1 expression is elevated compared to normal levels. This means that the mother has increased risk of having autistic child. Vitamin D is then be administered ˜1,000 IU per 11.3 kg per day. After 4 weeks of the vitamin D treatment, TPH1 expression levels are assayed in peripheral blood by ELISA analysis again. The levels of TPH1 have decreased to normal range. The vitamin D treatment will continue through pregnancy and breastfeeding. This implies the risk of having autistic child is reduced.

### Example 6. Determination of TPH1 in the Placenta

TPH1 expression levels are determined from placental tissue homogenate obtained from a pregnant woman by ELISA analysis. The results show that TPH1 expression is elevated compared to normal levels. This means that the mother has increased risk of having child with autistic spectrum disorder. Vitamin D is then administered to the pregnant woman at about 1,000 IU per 11.3 kg per day. After 4 weeks of the vitamin D treatment, TPH1 expression levels are assayed in placental tissue homogenate by ELISA analysis again. The levels of TPH1 have decreased to normal range. This implies the risk of having autistic child is reduced.

### Example 7. Determination of TPH1 Level in the Peripheral Tissue

TPH1 expression levels are determined from peripheral blood cells by ELISA analysis from a child less than two years old. The results show that TPH1 expression is elevated compared to normal levels. This implies this child has increased risk (high risk) of autistic spectrum disorder, schizophrenia, ADHD, bipolar, anti-social behavior disorder. The vitamin D/tryptophan/omega-3 formulation of Example 3 is administered daily to the young child. After 4 weeks of the treatment, TPH1 expression levels are assayed in peripheral blood by ELISA analysis again. The TPH1 levels now appear in the normal range. This implies the formulation is working and should be continued prophylactically for the prevention of all of these disorders.

1. A method of increasing brain serotonin level in an individual, comprising orally administering to the individual an effective amount of vitamin D and an effective amount of tryptophan, wherein the amount of the vitamin D is at least about 500 IU per day, and the amount of the tryptophan is at least about 100 mg per day.

2. A method of treating a brain dysfunction disorder in an individual, comprising orally administering to the individual an effective amount of vitamin D and an effective amount of tryptophan, wherein the amount of the vitamin D is at least about 500 IU per day, and the amount of the tryptophan is at least about 100 mg per day.

3. A method of improving pro-social behavior or cognitive function in an individual, comprising orally administering to the individual an effective amount of vitamin D and an effective amount of tryptophan, wherein the amount of the vitamin D is at least about 500 IU per day, and the amount of the tryptophan is at least about 100 mg per day.

4. The method of any one of claims 1-3, wherein the amount of the vitamin D is between about 500 IU per day to about 6000 IU per day.

5. The method of any one of claims 1-4, wherein the vitamin D and the tryptophan are administered simultaneously.

6. The method of claim 5, wherein the vitamin D and the tryptophan are administered in a single composition.

7. The method of any one of claims 1-6, further comprising administering to the individual one or more of: vitamin B6, BH4 (tetrahydrobiopterin), omega-3 long chain polyunsaturated fatty acid (LCPUFA), and iron.

8. The method of claim 7, further comprising administering to the individual LCPUFA.

9. The method of claim 8, wherein the amount of LCPUFA is at least about 500 mg.

10. The method of any one of claims 1-9, wherein the method further leads to reduced inflammation in the GI tract.

11. The method of any one of claims 1-10, wherein the method further leads to an increase of oxytocin level.

12. The method of any one of claims 1-11, wherein the individual is human.

13. The method of claim 12, wherein the individual is younger than 12 years old.

14. The method of claim 12, wherein the individual is a pregnant individual.

15. The method of any one of claims 1-14, wherein the individual has a high peripheral TPH1 level.

16. A composition comprising vitamin and amino acid, wherein at least about 5% of the vitamin in the composition is vitamin D, wherein at least about 30% of the amino acid in the composition is tryptophan.

17. The composition of claim 16, wherein the composition further comprises one or more of: vitamin B6, BH4, omega-3 long chain polyunsaturated fatty acid (LCPUFA), and iron.

18. The composition of claim 16 or 17, wherein the unit dose of vitamin D in the composition is at least about 500 IU.

19. The composition of claims 16-18, wherein the unit dose of tryptophan in the composition is at least about 0.5 grams.

20. A method of assessing the brain serotonin level in an individual, comprising determining the peripheral TPH1 level in the individual using an antibody recognizing TPH1, wherein a high TPH1 level is indicative that the individual has a low brain serotonin level.

21. A method of assessing the risk of developing a brain dysfunction disorder in an individual using an antibody recognizing TPH1 using an antibody recognizing TPH1, comprising determining the peripheral TPH1 level in the individual using an antibody recognizing TPH1, wherein a high TPH1 level is indicative that the individual has a low brain serotonin level.

22. The method of claim 20-21, further comprising administering to the individual an agent that increases brain serotonin level.

23. The methods of claim 20-22, further comprising administering to the individual an effective amount of vitamin D.

24. A method of assessing the placenta serotonin level in a pregnant individual, comprising determining the placenta TPH1 level in the individual, wherein a high TPH1 level is indicative that the individual has a high placenta serotonin level.

25. A method of assessing the risk of a pregnant individual having a child who develops a brain dysfunction disorder, comprising determining the placenta TPH1 level in the individual, wherein a high TPH1 level is indicative of the risk.

26. The method of claim 24 or 25, further comprising administering to the individual an effective amount of vitamin D.

27. A kit comprising a) an agent that increases brain serotonin level; and b) an agent for determining the level of TPH1.

28. The kit of claim 27, wherein the agent for determining the level of TPH1 is an antibody recognizing TPH1.
